(19) United States
(12) Patent Application Publication
(10) Pub. No.: US 2023/0201817 A1
(43) Pub. Date: Jun. 29, 2023

(54) MODULAR DRUG PRODUCTION SYSTEM

(71) Applicants: BioNTech Delivery Technologies GmbH, Halle (DE); BioNTech SE, Mainz (DE)

(72) Inventors: Christian Reinsch, Halle (DE); David Estapé Izquierdo, Basel (CH); Sierk Poetting, Munich (DE); Ugur Sahin, Mainz (DE); Alexander Muik, Seeheim-Jugenheim (DE); Asaf Poran, Stoneham, MA (US); Martin Lang, Bingen am Rhein (DE); Oliver Hennig, Mainz (DE); Karsten Pietron-Kattmann, Lohra (DE); Manfred Brunen, Marburg (DE); Rainer Kröner, Kirchhain (DE); Mario Büttner, Marburg (DE); Sven Stegmann, Munich (DE); Kristin Hoffmann, Ebsdorfergrund (DE)

(21) Appl. No.: 18/085,206

(22) Filed: Dec. 20, 2022

Related U.S. Application Data
(60) Provisional application No. 63/420,508, filed on Oct. 28, 2022.
(60) Provisional application No. 63/419,690, filed on Oct. 26, 2022.
(60) Provisional application No. 63/301,834, filed on Jan. 21, 2022.
(60) Provisional application No. 63/293,480, filed on Dec. 23, 2021.

(51) Int. Cl. B01L 1/00; G16B 30/00; G16B 50/30; G16B 20/20; A61K 39/215; B01F 25/25; B01D 61/14

(52) U.S. Cl. CPC B01L 1/52; A61K 39/215; B01D 61/145; B01D 61/147; B01D 61/149; B01F 25/25; G16B 20/20; G16B 30/00; G16B 50/30; A61K 2039/53

(57) ABSTRACT

A portable system for producing a formulation comprising lipid nanoparticle (LNP)-encapsulated RNA includes: a first sub-system comprising multiple drug substance formulation modules, the first sub-system comprising: a transcription module for forming an RNA solution via in vitro transcription; and a second sub-system operatively downstream of the first sub-system comprising multiple drug product formation modules, the second sub-system comprising: an LNP formulation module for producing a first RNA-LNP preparation from the RNA solution, wherein each of the transcription module and the LNP formulation module is contained within a separate standard shipping container.

### CROSS-REFERENCE TO RELATED APPLICATIONS

This application claims priority to U.S. Provisional Pat. Application No. 63/293,480, filed Dec. 23, 2021; U.S. Provisional Pat.Application No. 63/301,834, filed Jan. 21, 2022; U.S. Provisional Pat. Application No. 63/419,690, filed Oct. 26, 2022; and U.S. Provisional Pat. Application No. 63/420,508, filed Oct. 28, 2022, the title of each of which is “Modular Drug Production System,” and the content of each of which is incorporated herein by reference in its entirety.

### BACKGROUND

Nucleic acids represent an important therapeutic modality; lipid nanoparticle technologies have proven to be particularly useful for the delivery of nucleic acid therapeutics, specifically including RNA therapeutics. The ability to deliver lipid nanoparticles, nucleic acids, and/or drug products resulting therefrom, (as well as other drug products) in a time-sensitive manner is often constrained by drug product manufacturing capacity, as well as the ability to rapidly adapt drug product manufacturing to current needs.

### SUMMARY

The present disclosure provides technologies relating to modular drug product manufacturing including lipid nanoparticle (LNP) compositions, as well as other drug products. Quickly producing and delivering therapies and/or treatments to patients often involves challenges relating to but not limited to manufacturing capacity, manufacturing flexibility, the overall time required to produce and deliver a drug product to a patient, as well as other considerations. Often, local requirements relating to certification and approval of drugs categorize drug products differently depending on if they are produced within a country or imported from another country. For example, different regulatory approval hurdles may exist for drugs that are produced within a country than those that exist for drugs that are imported.

The present embodiments include a modular drug production system that may be shipped anywhere in the world that standard shipping (i.e., “overseas”) containers are able to be shipped. The modular drug production system of the present embodiments may be particularly useful for producing RNA-LNP drug products, but may also be useful for any number of other types of drug products. The modular drug production system of the present embodiments enables drugs to be produced within a country and/or localized region, thereby allowing for drug products that address localized outbreaks and viral strains to be manufactured, in the exact location in which they are needed. In addition, the modular drug production system of the present embodiments allows for an increase in the overall drug manufacturing capacity, and offloads the demand and/or pressure on centralized large scale drug manufacturing facilities to undertake smaller-scale production runs (i.e., to address regional and/or localized viral strains) to the possible detriment of large scale production (which is primarily focused on helping the greatest number people in the most efficient manner).

In one aspect, the present disclosed embodiments are directed to a biopharmaceutical manufacturing facility comprising at least one biopharmaceutical production unit, said biopharmaceutical production unit comprising two or more modularly arranged containment units containing one or more apparatus adaptable for (1) receipt and processing of active ingredient or active ingredient precursor, and pharmaceutically acceptable carrier or excipient, and resultant biopharmaceutical product production (2) biopharmaceutical product quality control, and (3) biopharmaceutical product filling and finishing, said two or more modularly arranged containment units being positioned and optionally interconnected for the inter-unit transfer of unfinished biopharmaceutical product and delivery of finished biopharmaceutical product.

In some embodiments, the facility comprises between one and ten production units (i.e., containers or modules), each of said production units comprising: (a) a first containment unit containing one or more apparatus adaptable for receipt and processing of active ingredient or active ingredient precursor, and pharmaceutically acceptable carrier or excipient, and resultant biopharmaceutical product production; (b) a second containment unit in communication with the first containment unit for the receipt of biopharmaceutical product and containing one or more apparatus adaptable for biopharmaceutical product quality control, and (3) a third containment unit in communication with the second containment unit for the receipt of quality control-evaluated biopharmaceutical product and containing one or more apparatus adaptable for filling and finishing of quality control-evaluated biopharmaceutical product and delivery of finished biopharmaceutical product.

In some embodiments, at least two of the modularly arranged containment units are horizontally arranged.

In some embodiments, the manufacturing facility is at least partially enclosed in a housing. In some embodiments, both the active ingredient and active ingredient precursor are each nucleotides, processing includes encapsulation of a processed nucleotide within a lipid nanoparticle and the resultant biopharmaceutical product is a nucleotide vaccine. In some embodiments, the active ingredient is mRNA.

In some embodiments, at least one of the containment units is an ISO container. In some embodiments, the one or more apparatus adaptable for receipt and processing of active ingredient or active ingredient precursor, and pharmaceutically acceptable carrier or excipient, and resultant biopharmaceutical product production include a bioreactor adaptable for receipt of DNA and the in vitro transcription of said DNA into mRNA. In some embodiments, the one or more apparatus adaptable for receipt and processing of active ingredient or active ingredient precursor, and pharmaceutically acceptable carrier or excipient, and resultant biopharmaceutical product production include purification apparatus configured for the removal of DNA impurities from the mRNA.

In some embodiments, the biopharmaceutical manufacturing facility produces from about 1 million to about 50 million doses of finished mRNA vaccine per year, or about 3,000 to about 170,000 doses of finished vaccine per day. In some embodiments, the apparatus adaptable for: (1) receipt and processing of active ingredient or active ingredient precursor, and pharmaceutically acceptable carrier or excipient, and resultant biopharmaceutical product production (2) biopharmaceutical product quality control, and (3) filling and finishing of quality control-evaluated biopharmaceutical product are in electronic communication with process control systems for remote operation. In some embodiments, the electronic communication is wireless; process control is at least partially automated; process control systems are controlled by one or more computers; and/or the one or more computers are remote from the facility.

In another aspect, the present disclosed embodiments are directed to a portable system for producing a formulation comprising lipid nanoparticle (LNP)-encapsulated RNA, the system comprising: a first sub-system comprising multiple drug substance formulation modules, the first sub-system comprising: a transcription module for forming an RNA solution via in vitro transcription (e.g., of a DNA template); and a second sub-system operatively downstream of the first sub-system comprising multiple drug product formation modules, the second sub-system comprising: an LNP formulation module for producing a first RNA-LNP preparation from the RNA solution, wherein each of the transcription module and the LNP formulation module is contained (or shipped to a site) within a separate standard shipping container.

In some embodiments, each separate standard shipping container comprises a width of about 8 ft (2.43 m), a height of about 8.5 ft (2.59 m) and a length from about 20 ft (6.06 m) to about 40 ft (12.12 m).

In some embodiments, the LNP formulation module comprises at least one impingement jet mixing unit. In some embodiments, the second sub-system further comprises a purification module disposed operatively downstream of the LNP formulation module, the purification module comprising at least one tangential flow filtration (TFF) unit, wherein the purification module is disposed within a separate standard shipping container. In some embodiments, wherein each of the first sub-system and the second sub-system comprises a bioburden reduction module disposed within a separate standard shipping container, each bioburden reduction module comprising a filtration unit comprising at least one filter with a pore size from about 0.05 µm to about 0.35 µm.

In another aspect, the present disclosed embodiments are directed to a portable system for producing a formulation comprising lipid nanoparticle (LNP)-encapsulated RNA, the system comprising: a first module comprising a transcription module for forming an RNA solution via in vitro transcription; a second module comprising a first purification module comprising a first tangential flow filtration (TFF) unit, the second module operatively downstream of the first module and receiving the RNA solution therefrom; a third module comprising a first bioburden reduction module, the third module operatively downstream of the second module; a fourth module comprising an LNP formulation module for producing a first RNA-LNP preparation from the RNA solution, the fourth module operatively downstream from the third module; a fifth module comprising a second purification module comprising a second tangential flow filtration (TFF) unit, the fifth module operatively downstream of the fourth module and receiving the first RNA-LNP preparation therefrom; and a sixth module comprising a second bioburden reduction module, the sixth module operatively downstream of the fifth module, wherein each of the first through sixth modules are disposed (and/or transported to a production site) within a separate standard shipping container.

In another aspect, the present disclosed embodiments are directed to a drug production system comprising: a drug substance module for producing at least one drug substance; a drug product module for producing a drug product at least partially from the at least one drug substance; wherein each of the drug substance module and the drug product module is disposed entirely in one or more portable shipping containers, and wherein the drug product is used to treat and/or vaccinate a patient within 1, 2, 4, 8, 12, 24, 48, and/or 72 hours of being produced.

In another aspect, the present disclosed embodiments are directed to a portable LNP formulation system for producing a first RNA-LNP preparation comprising: an impingement jet mixing unit, a first fluid conduit for delivering an RNA solution to the impingement jet mixing unit, the first fluid conduit fluidly connecting the impingement jet mixing unit to an RNA solution source external to the system; a second fluid conduit for delivering a lipid solution to the impingement jet mixing unit, the second fluid conduit fluidly connecting the impingement jet mixing unit to a lipid solution source external to the system; and a third fluid conduit for delivering the first RNA-LNP preparation to a downstream module external to the system, wherein the system is disposed (and/or transported to a production site) within a single standard shipping container.

In another aspect, the present disclosed embodiments are directed to a portable LNP formulation system for producing a first RNA-LNP preparation comprising: an impingement jet mixing unit; and a tangential flow filtration (TFF) unit coupled fluidly downstream of the impingement jet mixing unit, the TFF unit for performing at least one diafiltration step and at least one ultrafiltration step, wherein the system is disposed (and/or transported to a production site) within a single standard shipping container.

In some embodiments, the system includes a bioburden reduction unit coupled fluidly downstream of the TFF unit and contained within the standard shipping container.

In some embodiments, the system includes a first fluid conduit for delivering an RNA solution to the impingement jet mixing unit, the first fluid conduit fluidly connecting the impingement jet mixing unit to an RNA solution source external to the system; a second fluid conduit for delivering a lipid solution to the impingement jet mixing unit, the second fluid conduit fluidly connecting the impingement jet mixing unit to a lipid solution source external to the system; and a third fluid conduit for delivering a first RNA-LNP preparation to the TFF unit.

In another aspect, the present disclosed embodiments are directed to a drug production system comprising: a drug substance module for producing at least one drug substance; a drug product module for producing a drug product comprising the at least one drug substance; wherein each of the drug substance module and the drug product module are disposed entirely in one or more portable shipping containers.

In some embodiments, each of the drug substance module and the drug product module are disposed within at least three (3) portable shipping containers. In some embodiments, the drug product comprises at least one lipid nanoparticle (LNP). In some embodiments, the system includes at least one fill and finish module for disposing the drug product into at least one container.

In some embodiments, the combined power requirement of the drug substance module and the drug product module is in a range from about 200 kW to about 400 kW with an uninterrupted power requirement in a range from about 50 kW to about 100 kW.

In some embodiments, the combined footprint of the drug substance module and the drug product module encompasses an area of from about 500 square meters to about 1000 square meters.

In some embodiments, each of the drug substance module and the drug product module includes at least one airlock through materials and/or personnel must pass when entering an operations area within the respective drug substance module and drug product module.

In some embodiments, the system includes a quality control module including a PCR lab, an RNA/DNA lab, an environmental monitoring console, an HPLC lab, a cell culture lab, a general procedure lab, freezer monitoring equipment, a bioburden lab, a quality control storage area, a washing area, an endotoxin lab, and/or a gowning area.

Among other things, the present disclosure identifies the source of a problem that can be encountered with lipid nanoparticle compositions. Without wishing to be bound by any particular theory, the present disclosure proposes that failure to avoid, detect, or remove air from LNP compositions (e.g., nucleic acid-LNP compositions, and specifically RNA-LNP compositions) can have one or more detrimental effects including, for example, loss of colloidal stability, interference with ability to pass through filters, etc. Furthermore, the present disclosure provides insights that certain negative impacts may emerge and/or may become particularly impactful when relevant compositions are prepared at large scale.

The present disclosure provides technologies for improving LNP manufacturing, transport and/or storage. Those skilled in the art, reading the present disclosure, will appreciate the significance and applicable breadth of its teachings.

Among other things, those skilled in the art will appreciate the increasing significance of nucleic acid therapeutics (e.g, oligonucleotide therapeutics, as well as longer DNA and/or RNA therapeutics), including the transformative impact of RNA vacines during the COVID19 pandemic.

Those skilled in the art will further appreciate the importance of delivery technologies, and particularly of LNP delivery technologies, to the success of nucleic acid therapeutics, specifically including therapeutic RNAs e.g., therapeutic mRNAs.

In some embodiments, provided technologies are useful for manufacturing pharmaceutical-grade RNA therapeutics). In some embodiments, provided technologies may be particularly useful for large scale manufacturing of RNA therapeutics, e.g., ofpharmaceutical-grade RNA therapeutics.

Among other things, in some embodiments, the present disclosure identifies the source of one or more challenges that can be associated with manufacturing and/or maintaining certain LNP compositions, specifically including RNA-LNP compositions. Among other things, the present disclosure provides technologies that facilitate consistent manufacturing, for example, satisfying pre-determined in-process controls, and/or lot release specifications (e.g., high purity, integrity, potency, etc.). In some embodiments, the present disclosure provides robust manufacturing technologies for LNP (e.g., RNA-LNP) compositions, including technologies that can be performed at scale, while maintaining particular product attributes, such as high purity, integrity, stability (e.g., to transportation and/or storage) etc.. In some embodiments, relevant product attributes may be or include, for example, colloidal stability, particle size (and/or size distribution), LNP topology, amenability to further processing and/or formulation, effectiveness of delivery of encapsulated material from administered compositions, etc.

Among other things, in some embodiments, the present disclosure provides technologies for avoiding the introduction of air at various stages and/or processes associated with manufacturing LNP compositions. In some embodiments, manufacture described herein can comprise one or more of: lipid stock preparation, proparation of stock of an encapsulated agent (e.g., a nucleic acid agent such as an RNA agent), agent (e.g., nucleic acid, e.g., RNA)-LNP formulation, stabilization (e.g., by dilution), concentration, purification or separation (e.g., buffer exchange, and/or filtration), concentration adjustment addition of one or more excipients (e.g., cryoprotectant), aseptic filling, labelling, storage, and/or characterization of LNP-containing drug product and/or one or more components thereof, any or all of which may bes performed under pre-determined conditions and parameters that yield a large-scale mass throughput of drug product (e.g., as described herein) while maintaining product attributes (e.g., as described herein).

In some embodiments, technologies described herein can be utilized in different manufacturing scales. In some embodiments, technologies described herein can be utilized in parallel to further improve throughput capacity.

In some embodiments, technologies provided herein can utilize nucleic acid (e.g., RNA) manufactured in batch sizes within a range of about 0.01 g to about 500 g, about 0.01 g to about 10 g, about 1 g to about 10 g, about 10 g to about 500 g, about 10 g to about 300 g, about 10 g to about 200 g or about 30 g to about 60 g.

Among other things, in some embodiments, the present disclosure provides methods for characterizing LNP products (e.g., nucleic acid-LNP products such as RNA-LNP products) manufactured that are suitable for use in pharmaceutical products.

In some embodiments, the present disclosure, among other things, provides technologies (e.g., systems and/or methods) for reducing the introduction of air into drug product manufacturing processes, thereby reducing the likelihood of disrupting and/or otherwise impacting LNP characteristics including, for example, encapsulation (e.g., of nucleic acids and specifically of RNA) within lipid nanoparticles and/or stability (e.g, colloidal stability) of nanoparticle preparations, in each case particularly when at large scale.

Without wishing to be bound by any particular theory, the present disclosure proposes that, in some embodiments, the air-liquid interface results in disruption of one or more features of LNP structure - for example separation of PEG-lipids from other lipid components and/or or other alteration(s) that may cause collapse or instability of LNP colloids and/or of the LNPs within them/

In some embodiments, use of various systems and/or methodologies to prevent the introduction of air into the manufacturing process at one or more stages reduces the probability of such negatice impact(s). Without wishing to be bound by a particular theory, in some embodiments, in order to avoid the introduction of air, additional systems and/or process steps may be utilized in connection with at least three manufacturing steps including 1) impingement jet mixing, 2) tangential flow filtration (TFF), and 3) transporting of product (i.e., intermediate drug product being transported from one site to another, for example, not necessarily the final product). In some embodiments, in order to avoid the introduction of air, additional systems and/or process steps may be utilized in connection with a fourth step including the final fill and finishing processes. In some embodiments, an aqueous solution of RNA and a lipid solution are mixed under operating conditions sufficient to form a first LNP preparation such that substantially no air is introduced. In some embodiments, air bubbles are removed manually form transport bags via syringe. In some embodiments, one or more LNP preparations may include a buffer exchange configured such that substantially no air is introduced. In some embodiments, TFF may include the use of one or more jejunostomy tubes and/or one or more dip tubes configured to avoid introducing air into one or more LNP preparations.

In some embodiments, the present disclosure provides technologies (i. e., systems and methods) for producing lipid nanoparticle (LNP)-encapsulated RNA including: flowing an aqueous solution of RNA and a lipid solution independently into a mixing unit; and mixing, in the mixing unit, the aqueous solution of RNA and the lipid solution under operating conditions sufficient to form a first RNA-LNP preparation comprising LNP-encapsulated RNA, and to introduce substantially no air into the first RNA-LNP preparation. In some embodiments, the present disclosure provides technologies (i.e., systems and methods) for producing a formulation including lipid nanoparticle (LNP)-encapsulated RNA including: an impingement jet mixing unit configured to mix an aqueous solution including RNA and a lipid solution to produce a first RNA-LNP preparation comprising LNP-encapsulated RNA. The system may include: one or more tangential flow filtration units configured to exchange solvent and/or concentrate the first RNA-LNP preparation to produce a second RNA-LNP preparation; and one or more jejunostomy tubes in fluid communication with the impingement jet mixing unit and/or the one or more tangential flow filtration units, configured to transport liquid in the system while introducing substantially no air into the liquid.

Technologies described herein can be useful for manufacturing LNP compositions (e.g., of LNPs encapsulating nucleic acid, and in particular RNA such as, e.g., mRNA). In some embodiments, technologies described here can be useful for manufacturing LNP compositions (e.g., nucleic acid-LNPs, e.g., RNA-LNPs) for treatment and/or prevention of a disease, disorder, or condition (e.g., cancer, infectious diseases, diseases associates with protein deficiency, etc.).

In some embodiments, technologies described herein can be useful for manufacturing LNP compositions that comprise or deliver a nucleic acid encoding a polypeptide. In some embodiments, technologies described herein can be useful for manufacturing LNP for inducing an immune response to an antigen (e.g., an antigen encoded by a nucleic acid that is included in or delivered by an LNP composition).

In some embodiments, the present disclosure is directed to a system that includes at least one DNA sequencer for sequencing at least one local strain of a disease.

In some embodiments, the system includes at least one DNA synthesizer for creating at least one custom DNA molecule.

In some embodiments, the system includes at least one computing system for performing at least one of the following tasks: uploading sequence information describing at least one local strain of a disease to a public database, downloading sequence information describing the at least one local strain of a disease from the public database, downloading DNA synthesis data to be used for making a vaccine that targets the at least one local strain of a disease from the public database, and computing, based on the sequence information describing at least one local strain of a disease, a target strain upon which DNA synthesis data is based.

In another aspect, the present disclosed is directed to a method of producing a vaccine to treat a local strain of a disease, the method comprising: filtering genomic data for the disease by at least one location, thereby producing localized data; determining a target strain from the localized data; sending DNA synthesis instructions to a site within, or proximate to, the at least one location; producing, within, or proximate to, the at least one location, the vaccine to treat the local strain based on the DNA synthesis instructions.

In some embodiments, the disease is SARS-CoV-2.

In some embodiments, the method includes administering the vaccine and distributing the vaccine within, or proximate to, the at least one location.

In some embodiments, the method includes accessing a publically available database that houses the genomic data to be filtered.

In some embodiments, the method includes: sequencing, within, or proximate to, the at least one location, a sample of the local strain of the disease, thereby producing local strain sequence data; and uploading the local strain sequence data to a publically available database that houses the genomic data to be filtered.

In some embodiments, the method includes filtering the genomic data based on at least one of a range of dates and a lookback period. The range of dates and/or lookback period corresponds to a timeframe during which a localized outbreak of the disease occurred.

In some embodiments, the method includes assessing deviations between the localized data and a baseline variant of the disease.

In some embodiments, the method includes comparing the deviations for one or more subsets within the localized data.

In some embodiments, the method includes assessing a level of commonality of the deviations for the one or more subsets within the localized data.

In some embodiments, determining a target strain from the localized data includes determining a target strain based at least partially on the deviations between the localized data and the baseline variant of the disease.

In some embodiments, technologies described herein can be useful for manufacturing RNA-LNP compositions for treatment and/or prevention of coronavirus infection, e.g., SARS-CoV-2 infection, as described in Walsh et al. “RNA-based COVID-19 vaccine BNT162b2 selected for a pivotal efficacy study” medRxiv preprint (2020), which is online accessible at: https://doi.org/10.1101/2020.08.17.20176651; and Milligan et al. “Phase I/II study of COVID-19 RNA vaccine BNT162b1 in adults” Nature (2020 August), which is online accessible at: https://doi.org/10.1038/s41586-020-2639-4, the contents of each of which are incorporated by reference in their entirety.

### BRIEF DESCRIPTION OF THE DRAWING

FIG. 1 depicts an exemplary modular drug production system, according to aspects of the present disclosure.

FIG. 2 depicts an overview of an exemplary drug product manufacturing enterprise and/or process, according to aspects of the present disclosure.

FIG. 3 depicts an overview of an exemplary drug product manufacturing site and/or process, according to aspects of the present disclosure.

FIG. 4 depicts an overview of an exemplary drug product manufacturing site, according to aspects of the present disclosure.

FIG. 5 depicts an overview of an exemplary drug product manufacturing site and/or module, according to aspects of the present disclosure.

FIG. 6 depicts an overview of an exemplary drug product manufacturing site and/or module, according to aspects of the present disclosure.

FIG. 7 depicts an overview of an exemplary drug product quality control site and/or module, according to aspects of the present disclosure.

FIG. 8 depicts an overview of an exemplary drug product warehouse site and/or module, according to aspects of the present disclosure.

FIG. 9 depicts an overview of an exemplary drug product manufacturing site, according to aspects of the present disclosure.

FIG. 10 depicts an overview of an exemplary drug product manufacturing site, according to aspects of the present disclosure.

FIG. 11 depicts an overview of exemplary manufacturing process for a pharmaceutical-grade composition comprising RNA, according to aspects of the present disclosure.

FIG. 12 illustrates an overview of exemplary DNA template manufacture process via a PCR-based process, according to aspects of the present disclosure.

FIG. 13 illustrates an exemplary process for manufacturing LNP compositions, according to aspects of the present disclosure.

FIG. 14 depicts an overview of an exemplary drug product manufacturing site and/or module, according to aspects of the present disclosure.

FIG. 15 depicts an overview of an exemplary drug product manufacturing site and/or module, according to aspects of the present disclosure.

FIG. 16 depicts an overview of an exemplary drug product manufacturing site and/or module, according to aspects of the present disclosure.

FIG. 17 illustrates a process for making vaccines, according to aspects of the present disclosure.

FIG. 18 illustrates a process for making vaccines, according to aspects of the present disclosure.

FIG. 19 depicts an overview of an exemplary drug product manufacturing enterprise and/or process, according to aspects of the present disclosure.

FIG. 20 depicts an overview of an exemplary drug product manufacturing enterprise and/or process, according to aspects of the present disclosure.

FIG. 21 depicts an overview of an exemplary drug product manufacturing enterprise and/or process, according to aspects of the present disclosure.

### CERTAIN DEFINITIONS

About or Approximately: The term “about” or “approximately”, when used herein in reference to a value, refers to a value that is similar, in context to a stated reference value. In general, those skilled in the art, familiar with the context, will appreciate the relevant degree of variance encompassed by “about” or “approximately” in that context. For example, in some embodiments, the term “about” or “approximately” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, intradermal, transdermal, etc.), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may be intramuscular. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, is used to refer to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present disclosure is in a format selected from, but not limited to, intact IgA, IgG, IgE or IgM antibodies; bi- or multi- specific antibodies (e.g., Zybodies®, etc.); antibody fragments such as Fab fragments, Fab′ fragments, F(ab′)2 fragments, Fd′ fragments, Fd fragments, and isolated complementarity determining regions (CDRs) or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 95%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody agents can be made by the skilled person using methods and commercially available services and kits known in the art. For example, methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

Antibodies may be produced by standard techniques, for example by immunization with the appropriate polypeptide or portion(s) thereof, or by using a phage display library. If polyclonal antibodies are desired, a selected mammal (e.g., mouse, rabbit, goat, horse, etc.) is immunized with an immunogenic polypeptide bearing a desired epitope(s), optionally haptenized to another polypeptide. Depending on the host species, various adjuvants may be used to increase immunological response. Such adjuvants include, but are not limited to, Freund’s, mineral gels such as aluminum hydroxide, and surface-active substances such as lysolecithin, pluronic polyols, polyanions, peptides, oil emulsions, keyhole limpet hemocyanin, and dinitrophenol. Serum from the immunized animal is collected and treated according to known procedures. If serum containing polyclonal antibodies to the desired epitope contains antibodies to other antigens, the polyclonal antibodies can be purified by immunoaffinity chromatography or any other method known in the art. Techniques for producing and processing polyclonal antisera are well known in the art.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts - including where interacting entities or moieties are studied in isolation or in the context of more complex systems (e.g., while covalently or otherwise associated with a carrier entity and/or in a biological system or cell).

Bioreactor: The term “bioreactor” as used herein refers to a vessel used for in vitro transcription described herein. A bioreactor can be of any size so long as it is useful for in vitro transcription. For example, in some embodiments, a bioreactor can be at least 0.5 liter, including 1, 2, 3, 4, 5, 6, 7, 8, 9, 10, 15, 20, 25, 30, 35, 40, 45, 50 liters or more, or any volume in between. The internal conditions of the bioreactor, including, but not limited to pH and temperature, are typically controlled during in vitro transcription. The bioreactor can be composed of any material that is suitable for in vitro transcription under the conditions as described herein, including glass, plastic or metal. One of ordinary skill in the art will be aware of and will be able to choose suitable bioreactor volume for use in practicing in vitro transcription.

Cap: As used herein, the term “cap” refers to a structure comprising or essentially consisting of a nucleoside-5′-triphosphate that is typically joined to a 5′-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a N7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a DNA template, wherein, in addition to the GTP, an IVT system also contains a cap analog, e.g., as known in the art. Non-limiting examples of a cap analog include a m7GpppG cap analog or an N7-methyl-,2′-O-methyl-GpppG ARCA cap analog or an N7-methyl-,3′-O-methyl-GpppG ARCA cap analog, or any commercially available cap analogs, including, e.g., CleanCap (Trilink), EZ Cap, etc.. In some embodiments, a cap analog is or comprises a trinucleotide cap analog.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Complementary: As used herein, the term “complementary” is used in reference to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

Detecting: The term “detecting” is used broadly herein to include appropriate means of determining the presence or absence of an entity of interest or any form of measurement of an entity of interest in a sample. Thus, “detecting” may include determining, measuring, assessing, or assaying the presence or absence, level, amount, and/or location of an entity of interest. Quantitative and qualitative determinations, measurements or assessments are included, including semi-quantitative. Such determinations, measurements or assessments may be relative, for example when an entity of interest is being detected relative to a control reference, or absolute. As such, the term “quantifying” when used in the context of quantifying an entity of interest can refer to absolute or to relative quantification. Absolute quantification may be accomplished by correlating a detected level of an entity of interest to known control standards (e.g., through generation of a standard curve). Alternatively, relative quantification can be accomplished by comparison of detected levels or amounts between two or more different entities of interest to provide a relative quantification of each of the two or more different entities of interest, i.e., relative to each other.

Determine: Those of ordinary skill in the art, reading the present specification, will appreciate that a step of “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Encapsulate: The term “encapsulate” or “encapsulation” is used herein to refer to at least a portion of a component is enclosed or surrounded by another material or another component in a composition. In some embodiments, a component can be fully enclosed or surrounded by another material or another component in a composition.

Excipient: As used herein, the term “excipient” refers to a non-therapeutic agent that may be included in a pharmaceutical composition, for example to provide or contribute to a desired property or effect (e.g., desired consistency, delivery, and/or stabilizing effect, etc.). In some embodiments, suitable pharmaceutical excipients to be added to a LNP composition may include, for example, salts, starch, glucose, lactose, sucrose, gelatin, sodium chloride, glycerol, propylene, glycol, water, ethanol and the like.

Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or a single-stranded RNA (e.g., an mRNA) encodes a polypeptide if transcription and translation of mRNA corresponding to that gene produces the polypeptide in a cell or other biological system. In some embodiments, a coding region of a single-stranded RNA encoding a target polypeptide agent refers to a coding strand, the nucleotide sequence of which is identical to the mRNA sequence of such a target polypeptide agent. In some embodiments, a coding region of a single-stranded RNA encoding a target polypeptide agent refers to a non-coding strand of such a target polypeptide agent, which may be used as a template for transcription of a gene or cDNA.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fed-batch process: The term “fed-batch process” as used herein refers to a process in which one or more components are introduced into a vessel, e.g., a bioreactor, at some time subsequent to the beginning of a reaction. In some embodiments, one or more components are introduced by a fed-batch process to maintain its concentration low during a reaction. In some embodiments, one or more components are introduced by a fed-batch process to replenish what is depleted during a reaction.

Five prime untranslated region: As used herein, the terms “five prime untranslated region” or “5′ UTR” refer to a sequence of an mRNA molecule that begins at the transcription start site and ends one nucleotide (nt) before the start codon (usually AUG) of the coding region of an RNA.

Functional: As used herein, a “functional” biological molecule is a biological molecule in a form in which it exhibits a property and/or activity by which it is characterized. In some embodiments, a biological molecule may have two functions (i.e., bifunctional) or many functions (i.e., multifunctional).

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product. As used herein, the term “gene product” or “expression product” generally refers to an RNA transcribed from the gene (pre-and/or post-processing) or a polypeptide (pre- and/or post-modification) encoded by an RNA transcribed from the gene.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Host cell. As used herein, refers to a cell into which exogenous material (e.g., DNA such as recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a host cell is a human, monkey, ape, hamster, rat, or mouse cell. In some embodiments, a host cell is eukaryotic. For example, an eukaryotic host cell may be CHO (e.g., CHO Kl, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, or a cell line derived from an aforementioned cell.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improved, increased or reduced: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

In vitro: The term “in vitro” as used herein refers to events that occur in an artificial environment, e.g., in a test tube or reaction vessel (e.g., a bioreactor), in cell culture, etc., rather than within a multi-cellular organism.

In vitro transcription. As used herein, the term “in vitro transcription” or “IVT” refers to the process whereby transcription occurs in vitro in a non-cellular system to produce a synthetic RNA product for use in various applications, including, e.g., production of protein or polypeptides. Such synthetic RNA products can be translated in vitro or introduced directly into cells, where they can be translated. Such synthetic RNA products include, e.g., but not limited to mRNAs, antisense RNA molecules, shRNA molecules, long non-coding RNA molecules, ribozymes, aptamers, guide RNAs (e.g., for CRISPR), ribosomal RNAs, small nuclear RNAs, small nucleolar RNAs, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In vitro transcription RNA composition. As used herein, the term “in vitro transcription RNA composition” refers to a composition comprising target RNA synthesized by in vitro transcription. In some embodiments, such a composition can comprise excess in vitro transcription reagents (including, e.g., ribonucleotides and/or capping agents), nucleic acids or fragments thereof such as DNA templates or fragments thereof, polypeptides or fragments thereof such as recombinant enzymes or host cell proteins or fragments thereof, and/or other impurities. In some embodiments, an in vitro transcription RNA composition may have been treated and/or processed prior to a purification processes that ultimately produces an RNA transcript preparation comprising RNA transcript at a desired concentration in an appropriate buffer for formulation and/or further manufacturing and/or processing. For example, in some embodiments, an in vitro transcription RNA composition may have been treated to remove or digest DNA template (e.g., using a DNase). In some embodiments, an in vitro transcription RNA composition may have been treated to remove or digest polypeptides (e.g., enzymes such as RNA polymerases, RNase inhibitors, etc.) present in an in vitro transcription reaction (e.g., using a protease).

In vivo: As used herein, the term “in vivo” refers to events that occur within a multi-cellular organism, such as a human and a non-human animal.

Nanoparticle: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, a nanoparticle has a diameter of less than 80 nm as defined by the National Institutes of Health. In some embodiments, a nanoparticle comprises one or more enclosed compartments, separated from the bulk solution by a membrane, which surrounds and encloses a space or compartment.

Nucleic acid/ Polynucleotide: As used herein, the term “nucleic acid” refers to a polymer of at least 2 nucleotides or more, including, e.g., at least 3 nucleotides, at least 4 nucleotides, at least 5 nucleotides, at least 6 nucleotides, at least 7 nucleotides, at least 8 nucleotides, at least 9 nucleotides, at least 10 nucleotides, or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, 1-methyl-pseudouridine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Pharmaceutical grade: The term “pharmaceutical grade” as used herein refers to standards for chemical and biological drug substances, drug products, dosage forms, compounded preparations, excipients, medical devices, and dietary supplements, established by a recognized national or regional pharmacopeia (e.g., The United States Pharmacopeia and The Formulary (USP-NF)).

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics). In some embodiments, a polypeptide may be or comprise an enzyme. In some embodiments, a polypeptide may be or comprise a polypeptide antigen. In some embodiments, a polypeptide may be or comprise an antibody agent. In some embodiments a polypeptide may be or comprise a cytokine.

Pure or Purified: As used herein, an agent or entity is “pure” or “purified” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure in a preparation.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid / Polynucleotide” above. An RNA can be a regulatory RNA (e.g., siRNA, microRNA, etc.), or a messenger RNA (mRNA). In some embodiments, an RNA is a mRNA. In some embodiments, where an RNA is a mRNA, a RNA typically comprises at its 3′ end a poly(A) region. In some embodiments where an RNA is a mRNA, an RNA typically comprises at its 5′ end, an art-recognized cap structure, e.g., for recognizing and attachment of a mRNA to a ribosome to initiate translation. In some embodiments, an RNA is a synthetic RNA. Synthetic RNAs include RNAs that are synthesized in vitro (e.g., by enzymatic synthesis methods and/or by chemical synthesis methods). In some embodiments, an RNA is a single-stranded RNA. In some embodiments, a single-stranded RNA may comprise self-complementary elements and/or may establish a secondary and/or tertiary structure. One of ordinary skill in the art will understand that when a single-stranded RNA is referred to as “encoding,” it can mean that it comprises a nucleic acid sequence that itself encodes or that it comprises a complement of the nucleic acid sequence that encodes. In some embodiments, a single-stranded RNA can be a self-amplifying RNA (also known as self-replicating RNA).

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

RNA polymerase: As used herein, the term “RNA polymerase” refers to an enzyme that catalyzes polyribonucleotide synthesis by addition of ribonucleotide units to a nucleotide chain using DNA or RNA as a template. The term refers to either a complete enzyme as it occurs in nature, or an isolated, active catalytic or functional domain, or fragment thereof. In some embodiments, an RNA polymerase enzyme initiates synthesis at the 3′-end of a primer or a nucleic acid strand, or at a promoter sequence, and proceeds in the 5′-direction along the target nucleic acid to synthesize a strand complementary to the target nucleic acid until synthesis terminates.

RNA transcript preparation. The term “RNA transcript preparation” as used herein refers to a preparation comprising RNA transcript that is purified from an in vitro transcription RNA composition described herein. In some embodiments, an RNA transcript preparation is a preparation comprising pharmaceutical-grade RNA transcript. In some embodiments, an RNA transcript preparation is a preparation comprising RNA transcript, which its one or more product quality attributes are characterized and determined to meet a release and/or acceptance criteria (e.g., as described herein). Examples of such product quality attributes include, but are not limited to appearance, RNA length, identity of drug substance as RNA, RNA integrity, RNA sequence, RNA concentration, pH, osmolality, residual DNA template, residual double stranded RNA, bacterial endotoxins, bioburden, and combinations thereof.

Room temperature: As used herein, the term “room temperature” refers to an ambient temperature. In some embodiments, a room temperature is about 18° C.-30° C., e.g., about 18° C.-25° C., or about 20° C.-25° C., or about 20-30° C., or about 23-27° C. or about 25° C.

Sample: As used herein, the term “sample” typically refers to an aliquot of material obtained or derived from a source of interest, e.g., as described herein. In some embodiments, a source of interest is a biological or environmental source. In some embodiments, a source of interest may be or comprise a cell or an organism, such as a microbe, a plant, or an animal (e.g., a mouse). In some embodiments, a source of interest is or comprises biological tissue or fluid. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a sample is or comprises cells obtained from a subject. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Stable: The term “stable,” when applied to nucleic acids and/or compositions comprising nucleic acids, e.g., encapsulated in lipid nanoparticles, means that such nucleic acids and/or compositions maintain one or more aspects of their characteristics (e.g., physical and/or structural characteristics, function, and/or activity) over a period of time under a designated set of conditions (e.g., pH, temperature, light, relative humidity, etc.). In some embodiments, such stability is maintained over a period of time of at least about one hour; in some embodiments, such stability is maintained over a period of time of about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, such stability is maintained over a period of time within the range of about one (1) day to about twenty-four (24) months, about two (2) weeks to about twelve (12) months, about two (2) months to about five (5) months, etc. In some embodiments, such stability is maintained under an ambient condition (e.g., at room temperature and ambient pressure). In some embodiments, such stability is maintained under a physiological condition (e.g., in vivo or at about 37° C. for example in serum or in phosphate buffered saline). In some embodiments, such stability is maintained under cold storage (e.g., at or below about 4° C., including, e.g., -20° C., or -70° C.). In some embodiments, such stability is maintained when nucleic acids and/or compositions comprising the same are protected from light (e.g., maintaining in the dark).

As an example, in some embodiments, the term “stable” is used in reference to a nanoparticle composition (e.g., a lipid nanoparticle composition). In such embodiments, a stable nanoparticle composition (e.g., a stable nanoparticle composition) and/or component(s) thereof maintain one or more aspects of its characteristics (e.g., physical and/or structural characteristics, function(s), and/or activity) over a period of time under a designated set of conditions. For example, in some embodiments, a stable nanoparticle composition (e.g., a lipid nanoparticle composition) is characterized in that average particle size, particle size distribution, and/or polydispersity of nanoparticles is substantially maintained (e.g., within 10% or less, as compared to the initial characteristic(s)) over a period of time (e.g., as described herein) under a designated set of conditions (e.g., as described herein). In some embodiments, a stable nanoparticle composition (e.g., a lipid nanoparticle composition) is characterized in that no detectable amount of degradation products (e.g., associated with hydrolysis and/or enzymatic digestion) is present after it is maintained under a designated set of conditions (e.g., as described herein) over a period of time.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Three prime untranslated region: As used herein, the terms “three prime untranslated region” or “3′ UTR” refer to the sequence of an mRNA molecule that begins following the stop codon of the coding region of an open reading frame sequence. In some embodiments, the 3′ UTR begins immediately after the stop codon of the coding region of an open reading frame sequence. In other embodiments, the 3′ UTR does not begin immediately after stop codon of the coding region of an open reading frame sequence.

Threshold level (e.g., acceptance criteria): As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g. a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Vector: As used herein, the term “vector” refers to a nucleic acid molecule capable of transporting another nucleic acid to which it has been linked. One type of vector is a “plasmid”, which refers to a circular double stranded DNA into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer’s specifications or as commonly accomplished in the art or as described herein. The foregoing techniques and procedures may be generally performed according to conventional methods well known in the art and as described in various general and more specific references that are cited and discussed throughout the present specification. See e.g., Green and Sambrook, Molecular Cloning: A Laboratory Manual (4th ed., Cold Spring Harbor Laboratory Press, Cold Spring Harbor, N.Y. (2012)), which is incorporated herein by reference for any purpose.

### DETAILED DESCRIPTION OF CERTAIN EMBODIMENTS

Nucleic acid therapeutics, and particularly RNA therapeutics have repersent a particularly promising class of therapies for treatment and prevention of various diseases such as cancer, infectious diseases, and/or diseases or disorders associated with overabundance or deficiency in certain proteins.

RNA therapeutics in particular provide remarkably effective as vaccines to address the COVID19 pandemic. Particularly given the promise of this technology, and its adaptability to a wide variety of clinical contexts, including massively large scale (e.g., vaccination and/or treatment on a global scale such as is under development for SARS-CoV-2), improvements to manufacturing technologies, especially those applicable to large-scale production, are especially valuable.

Development of effective delivery technologies has been central to the success of nucleic acid therapeutics, and lipid nanoparticle technologies have proven to be particularly effective (reviewed in, for example, Cullis et al. Molecular Therapy 25:1467, Jul. 5, 2017; See also, U.S. Pat. 8058069), specifically including for RNA therapeutics (reviewed in, for example, Hou et al., Nat. Rev. Mater doi.org/10.1038/s41578-021-00358-0, Aug. 10, 2021).

Technologies provided herein are useful, among other things, to achieve particularly effective and/or efficient production, e.g., on commercial scale and/or under commercial conditions, of pharmaceutical grade LNP preparations and/or compositions (e.g., nucleic acid-LNP preparations, and specifically RNA-LNP preparations). For example, in various embodiments, provided technologies permit and/or facilitate achievement of requirements unique to pharmaceutical-grade (and/or scale) production such as, for example, batch size and/or rate of production, pre-determined in-process controls and/or lot release specifications (e.g., high purity, integrity, potency, and/or stability, etc.), etc.

The present disclosure provides technologies for manufacturing LNP compositions (e.g., including RNA, e.g., therapeutic RNA such as therapeutic mRNA). In some embodiments, provided technologies are useful for manufacturing pharmaceutical-grade RNA-LNP therapeutics.

In some embodiments, provided technologies are useful for large scale manufacturing of LNP (e.g., nucleic acid-LNP, e.g., RNA-LNP) therapeutics, e.g., pharmaceutical-grade therapeutics. For example, in some such embodiments, technologies provided herein can be used to produce a pharmaceutical-grade batch throughput of at least 10,000 vials of LNP(e.g., nucleic acid-LNP, e.g., RNA-LNP) therapeutics (including, e.g., at least 20,000 vials, at least 30,000 vials, at least 40,000 vials, at least 50,000 vials, at least 60,000 vials, at least 70,000 vials, at least 80,000 vials, at least 90,000 vials, at least 100,000 vials, at least 200,000 vials, at least 300,000 vials, at least 400,000 vials, at least 500,000 vials, or more). For example, in some such embodiments, technologies provided herein can be used to produce a pharmaceutical-grade batch throughput of at least 50 L of LNP(e.g., nucleic acid-LNP, e.g., RNA-LNP) therapeutics (including e.g., at least 50 L, at least 60 L, at least 70 L, at least 80 L, at least 100 L, at least 110 L, at least 120 L, at least 130 L, at least 140 L, at least 150 L or more. In some embodiments, each vial can comprise a RNA drug product in an amount of 0.01 mg to 0.5 mg (e.g., 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg).

Technologies described herein can be useful for manufacturing LNP (e.g., nucleic acid-LNP, e.g., RNA-LNP) compositions for treatment and/or prevention of a disease, disorder, or condition (e.g., cancer, infectious diseases, diseases associates with protein deficiency, etc.). In some embodiments, technologies described herein can be useful for manufacturing LNP (e.g., nucleic acid-LNP, e.g., RNA-LNP) compositions that comprise or deliver (e.g., by comprising and/or delivering a nucleic acid, such as an RNA, that encodes it) a polypeptide.

In some particular embodiments, technologies described herein can be useful for manufacturing LNP (e.g., nucleic acid-LNP, e.g., RNA-LNP) compositions for inducing an immune response to an antigen. In some embodiments, technologies described herein can be useful for manufacturing LNP(e.g., nucleic acid-LNP, e.g., RNA-LNP) compositions for treatment and/or prevention of coronavirus infection, e.g., SARS-CoV-2 infection, as described in Walsh et al. “RNA-based COVID-19 vaccine BNT162b2 selected for a pivotal efficacy study” medRxiv preprint (2020), which is online accessible at: https://doi.org/10.1101/2020.08.17.20176651; and Milligan et al. “Phase I/II study of COVID-19 RNA vaccine BNT162b1 in adults” Nature (2020 August), which is online accessible at: https://doi.org/10.1038/s41586-020-2639-4, the contents of each of which are incorporated by reference in their entirety.

### Lipid Nanoparticles

Those skilled in the art are aware that lipid nanoparticles have achieved successful clinical delivery of a wide range of therapeutic agents including, for example, small molecules, and various nucleic acids - e.g., oligonucleotides, siRNAs, and mRNAs (reviewed, for example, in Hu et al., Nat. Rev. Mater. https://doi.org/10.1038/s41578-021-00358-0, Aug. 10, 2021).

Various routes of administration for lipid nanoparticle compositions have been proposed and/or tested; those skilled in the art will be aware of appropriate routes for particular compositions (e.g., depending on agent being delivered). To give but a few examples, in some embodiments, LNPs are parenterally administered; most clinical studies have utilized parenteral administration, and particularly intravenous, subcutaneous, intradermal, intravitreal, intratumoral, or intramuscular injection. Intrautero injection has also been described. In some embodiments, topical administration is utilized. In some embodiments, intranasal administration is utilized.

In some embodiments, administered LNPs are delivered to or accumulate in the liver. Given that the liver is naturally effective at producing and secreting proteins, liver delivery can prove useful for achieving delivery of an LNP-encapsulated agent (and/or, in the case of a nucleic acid agent such as an RNA agent, a polypeptide encoded thereby) into the bloodstream. Such liver delivery has been proposed to be particularly useful, for example, for expression of proteins that are missing in certain metabolic or hematological disorders, or that are effective in provoking immune responses (e.g., particularly antibody responses), for example against infectious agents or cancer cells.

In some embodiments, administered LNPs are delivered to and/or taken up by antigen-presenting cells (e.g., as may be present in skin, muscle, mucosal tissues, etc.); such administration may be particularly useful or effective for induction of T cell immunity (e.g., for treatment of infectious diseases and/or cancers).

In various embodiments, lipid nanoparticles can have an average size (e.g., mean diameter) of about 30 nm to about 150 nm, about 40 nm to about 150 nm, about 50 nm to about 150 nm, about 50 nm to about 130 nm, about 50 nm to about 110 nm, about 50 nm to about 100 nm, about 50 to about 90 nm, or about 60 nm to about 80 nm, or about 60 nm to about 70 nm. In some embodiments, lipid nanoparticles that may be useful in accordance with the present disclosure can have an average size (e.g., mean diameter) of about 50 nm to about 100 nm. In some embodiments, lipid nanoparticles may have an average size (e.g., mean diameter) of less than 80 nm, less than 75 nm, less than 70 nm, less than 65 nm, less than 60 nm, less than 55 nm, less than 50 nm, or less than 45 nm. In some embodiments, lipid nanoparticles that may be useful in accordance with the present disclosure can have an average size (e.g., mean diameter) of about 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In some embodiments, lipids that form lipid nanoparticles described herein comprise: a polymer-conjugated lipid; a cationic lipid; and a helper neutral lipid. In some such embodiments, total polymer-conjugated lipid may be present in about 0.5-5 mol%, about 0.7-3.5 mol%, about 1-2.5 mol%, about 1.5-2 mol%, or about 1.5-1.8 mol% of the total lipids. In some embodiments, total polymer-conjugated lipid may be present in about 1-2.5 mol% of the total lipids. In some embodiments, the molar ratio of total cationic lipid to total polymer-conjugated lipid (e.g., PEG-conjugated lipid) may be about 100:1 to about 20:1, or about 50:1 to about 20:1, or about 40:1 to about 20:1, or about 35:1 to about 25:1. In some embodiments, the molar ratio of total cationic lipid to total polymer-conjugated lipid may be about 35:1 to about 25:1.

In some embodiments involving a polymer-conjugated lipid, a cationic lipid, and a helper neutral lipid in lipid nanoparticles described herein, total cationic lipid is present in about 35-65 mol%, about 40-60 mol%, about 41-49 mol%, about 41-48 mol%, about 42-48 mol%, about 43-48 mol%, about 44-48 mol%, about 45-48 mol%, or about 46-49 mol% of the total lipids. In certain embodiments, total cationic lipid is present in about 47.0, 47.1, 47.2, 47.3, 47.4, 47.5, 47.6, 47.7, 47.8, 47.9 or 48.0 mol% of the total lipids.

In some embodiments involving a polymer-conjugated lipid, a cationic lipid, and a helper neutral lipid in lipid nanoparticles described herein, total neutral lipid is present in about 35-65 mol%, about 40-60 mol%, about 45-55 mol%, or about 47-52 mol% of the total lipids. In some embodiments, total neutral lipid is present in 35-65 mol% of the total lipids. In some embodiments, total non-steroid neutral lipid (e.g., DPSC) is present in about 5-15 mol%, about 7-13 mol%, or 9-11 mol% of the total lipids. In some embodiments, total non-steroid neutral lipid is present in about 9.5, 10 or 10.5 mol% of the total lipids. In some embodiments, the molar ratio of the total cationic lipid to the non-steroid neutral lipid ranges from about 4.1: 1.0 to about 4.9: 1.0, from about 4.5: 1.0 to about 4.8: 1.0, or from about 4.7: 1.0 to 4.8: 1.0. In some embodiments, total steroid neutral lipid (e.g., cholesterol) is present in about 35- 50 mol%, about 39-49 mol%, about 39-46 mol%, about 39- 44 mol%, or about 39-42 mol% of the total lipids. In certain embodiments, total steroid neutral lipid (e.g., cholesterol) is present in about 39, 40, 41, 42, 43, 44, 45, or 46 mol% of the total lipids. In certain embodiments, the molar ratio of total cationic lipid to total steroid neutral lipid is about 1.5:1 to 1: 1.2, or about 1.2: 1 to 1: 1.2.

In some embodiments, a lipid composition comprising a cationic lipid, a polymer-conjugated lipid, and a neutral lipid can have individual lipids present in certain molar percents of the total lipids, or in certain molar ratios (relative to each other) as described in WO 2018/081480, the entire contents of each of which are incorporated herein by reference for the purposes described herein.

In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2.5 mol% of the total lipids; the cationic lipid is present in 35-65 mol% of the total lipids; and the neutral lipid is present in 35-65 mol% of the total lipids. In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2 mol% of the total lipids; the cationic lipid is present in 45-48.5 mol% of the total lipids; and the neutral lipid is present in 45-55 mol% of the total lipids. In some embodiments, lipids that form the lipid nanoparticles comprise: a polymer-conjugated lipid (e.g., PEG-conjugated lipid); a cationic lipid; and a neutral lipid comprising a non-steroid neutral lipid and a steroid neutral lipid, wherein the polymer-conjugated lipid is present in about 1-2 mol% of the total lipids; the cationic lipid is present in 45-48.5 mol% of the total lipids; the non-steroid neutral lipid is present in 9-11 mol% of the total lipids; and the steroid neutral lipid is present in about 36-44 mol% of the total lipids. In many of such embodiments, a PEG-conjugated lipid is or comprises a structure

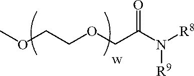

as described in WO 2017/075531 (also described above), or a derivative thereof. In some embodiments, a PEG-conjugated lipid is or comprises 2-[(polyethylene glycol)-2000]-N,N-ditetradecylacetamide. In many of such embodiments, a cationic lipid is or comprises a chemical structure selected from I-1 to I-10 of Table 1 herein or a derivative thereof. In some embodiments, a cationic lipid is or comprises ((4-hydroxybutyl)azanediyl)bis(hexane-6,1-diyl)bis(2-hexyldecanoate). In many of such embodiments, a neutral lipid comprises DSPC and cholesterol, wherein DSPC is a non-steroid neutral lipid and cholesterol is a steroid neutral lipid.

In some embodiments, lipid nanoparticles include one or more cationic lipids (e.g., ones described herein). In some embodiments, cationic lipid nanoparticles may comprise at least one cationic lipid, at least one polymer-conjugated lipid, and at least one helper lipid (e.g., at least one neutral lipid).

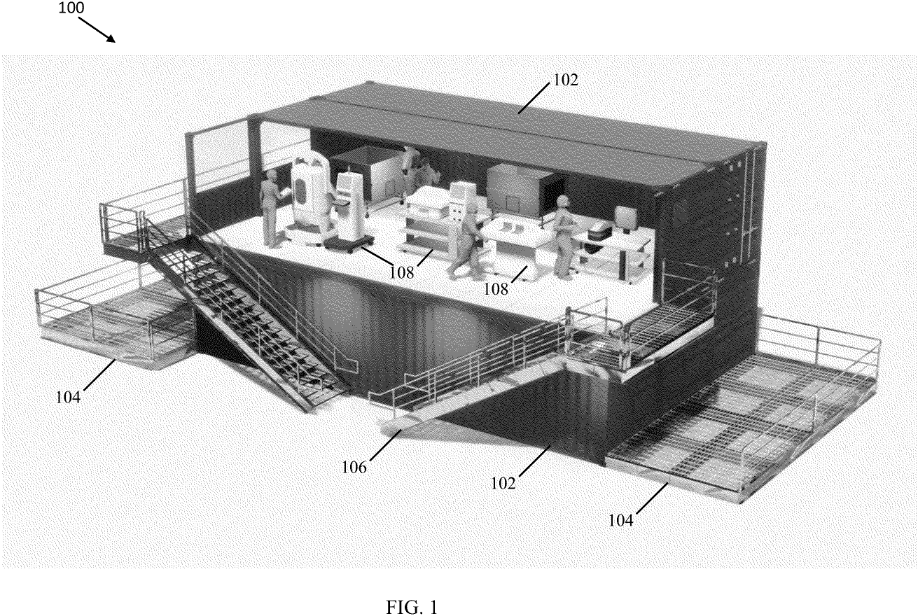

FIG. 1 depicts an exemplary modular drug production system 100, according to aspects of the present disclosure. In the embodiment of FIG. 1, the system 100 may include a total of five or six shipping containers 102 with three shipping containers 102 on the bottom and another two or three shipping containers 102 stacked on top of the bottom 3. The system 100 may include various external platforms 104 and/or staircases 106 as necessary to allow accessibility of personnel within the system 100. The system 100 includes various equipment 108 required for drug production. The equipment 108 may include tanks, filtration equipment, mixers, valves, sensors, process control and testing equipment (including computers), as well as other equipment. In some embodiments, the system 100 may include HVAC (heating, ventilation, and air conditioning) equipment to maintain the system 100 within a desired temperature range (for example, from about 15° C. to about 25° C., and/or from about 10° C. to about 30° C., as well as other suitable temperature ranges). In one or more embodiments, the system may include a first set of three containers 102 stacked on top of a second set of three containers 102 with the first set of three containers 102 containing a drug substance production module and/or a drug product production module, and with the second set of three containers containing HVAC equipment. In some embodiments, the HVAC equipment may be contained on the top level with the drug substance production module and/or a drug product production module contained on the bottom level. In some embodiments, the relative positions may be reversed (i.e., with the HVAC equipment on the bottom).

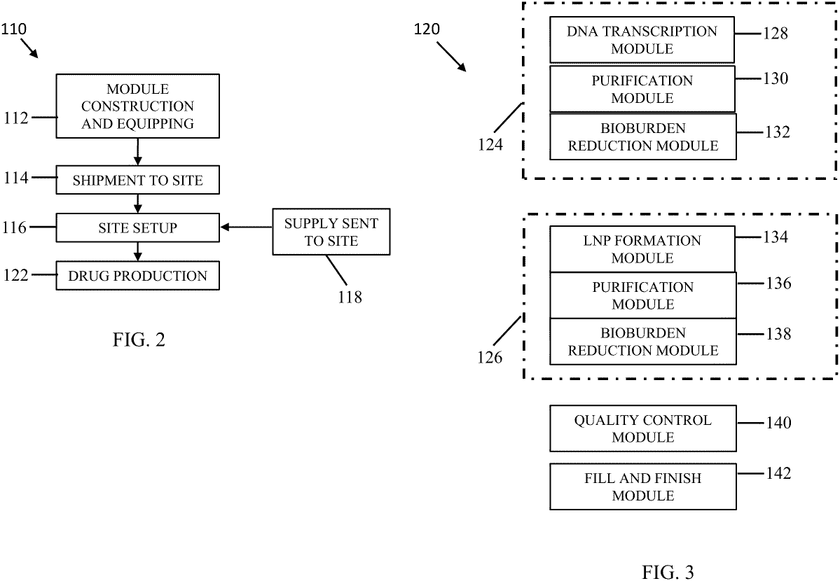

FIG. 2 depicts an overview of an exemplary drug product manufacturing enterprise 110 and/or process, according to aspects of the present disclosure. In the embodiment of FIG. 2, the enterprise 110 and/or process 110 may include constructing modules and equipping the modules (i.e., containers 102) at step 112 at a production facility (for example, at a BioNTech facility in Germany, and/or at one or more contractor and/or partner facilities located at various locations). At step 114, the enterprise 110 and/or process 110 may include shipping the constructed containers 102 and/or equipment to a drug production site. At step 116, the enterprise 110 and/or process 110 may include setting up the contracted modules (i.e.m containers 102) and/or equipment at the drug production site. At step 118, the enterprise 110 and/or process 110 may include shipping supplies (i.e., process input materials that are needed for producing drug substances and/or drug products) to the drug production site. In some embodiments, step 118 may occur concurrent with step 114 (that is, process input supplies are shipped to the site with the containers 102). At step 122, the enterprise 110 and/or process 110 may include initiating the production of drug substances and/or drug products.

FIG. 3 depicts an overview of an exemplary drug product manufacturing site, system and/or process 120, according to aspects of the present disclosure. In the embodiment of FIG. 3, the system 120 may generally include 8 containers 102. In some embodiments, the system 120 may include less than 8 containers 102 and/or more than 8 containers 102. The system 120 may include a first group of one or more containers 102 dedicated to drug substance production 124 as well as a second group of one or more containers 102 dedicated to drug product production 126. In addition, the system 120 may include a quality control module (and/or container) 140 as well as a fill and finish module (and/or container) 142. The first group of one or more containers 102 dedicated to drug substance production 124 may include a DNA transcription module 128 (or container), a first purification module 130 (or container), and a first bioburden reduction module 132 (or container). The second group of one or more containers 102 dedicated to drug product production 126 may include an LNP formation module 134 (or container), a second purification module 136 (or container), and a second bioburden reduction module 138 (or container). Each module or container may receive inputs from the previous or adjacent module. For example, the LNP formation module 134 may receive drug substance as an input from the first bioburden reduction module 132.

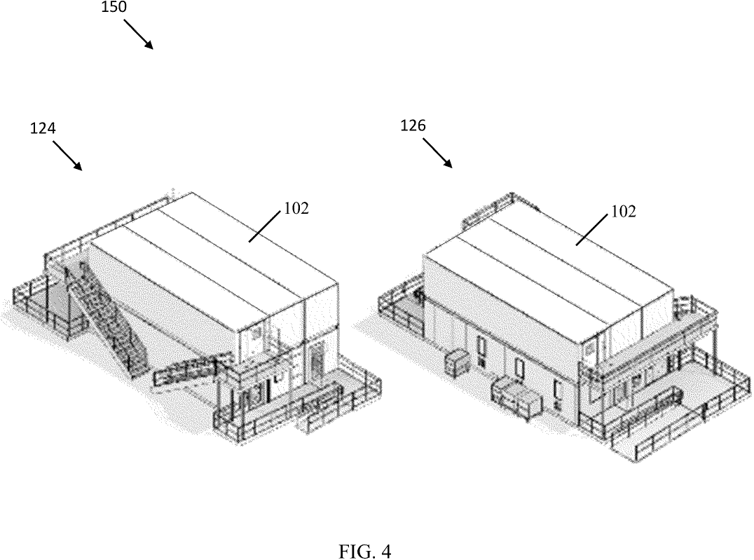

FIG. 4 depicts an overview of an exemplary drug product manufacturing site 150, according to aspects of the present disclosure. In the embodiment of FIG. 4, the site 150 may include the a first group of one or more containers 102 dedicated to drug substance production 124 as well as the second group of one or more containers 102 dedicated to drug product production 126. Each of the first and second groups 124, 126 may include six containers 102, with three of the six containers of each group housing HVAC equipment (for example on the first level or on the second level), as well as three of the six containers housing process modules, as shown in FIG. 3 (i.e., the first group three containers 124 housing the DNA transcription module 128, the first purification module 130 , and the first bioburden reduction module 132, and the second group of three containers 126 including the LNP formation module 134, the second purification module 136, and the second bioburden reduction module 138). In some embodiments, the site 150 may include different numbers of containers, and may include other modules as discussed herein (for example, a quality control module 140, a fill and finish module 142, and other modules as described herein). In some embodiments, the first and second groups 124, 126 may encompass a total area (or footprint (for example, a site footprint)) of about 800 m² or less, and may be able to produce approximately 50 million doses of vaccine per year.

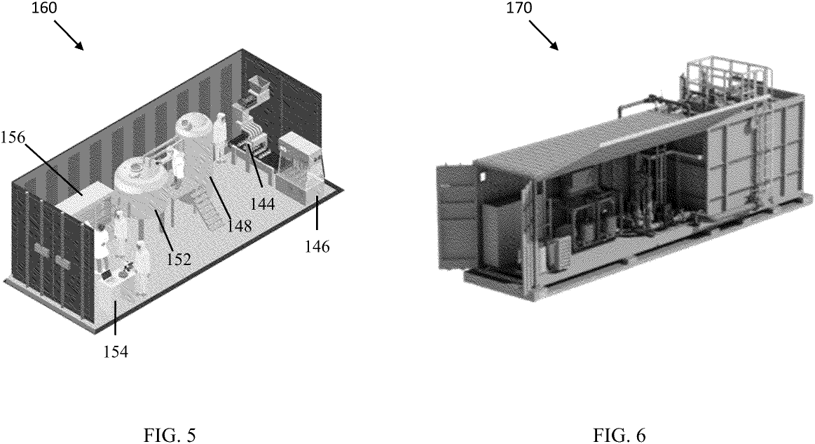

FIGS. 5 and 6 depict an overview of an exemplary drug product manufacturing site and/or module 160, according to aspects of the present disclosure. In the embodiment of FIG. 5, the module 160 may be or include any of the individual modules 128, 130, 132, 124, 126, 128, 140, and/or 142 including combinations thereof, as shown in FIG. 3, and as described herein. Referring to FIGS. 5 and 6, in some embodiments, the module 160 may include all of the associated equipment required for drug substance and/or drug product production in a single container (for example, for use in medium-scale, small-scale, and/or micro-scale production applications). For example, module 160 may be installed at a hospital or other location where individualize treatment and/or therapies are required in a time-sensitive setting, and on a relatively small scale. Module 160 may include equipment 144 for receiving and/or processing drug substance (and/or other process input supplies), tanks 148 and/or mixing equipment 148 (for example, for LNP (i.e., RNA-LNP production)) filtration equipment 152, process control equipment 154, fill and finish equipment 146, and/or storage or refrigeration equipment 156. In the context of larger scale production, certain equipment, such as mixing and filtration equipment, is only commercially available at fixed sizes, thereby partially dictating the size at which drug production can occur. At smaller scales, there may be vastly greater availability of equipment in increment sizes, meaning that the entire drug production (include drug substance and drug product manufacturing) can feasibly fit within a single shipping container 102. In addition, certain process steps or modules such as freezing and/or warehousing may not be required when the drug product is being delivered to (i.e., administered to) the patient as soon as it is ready. In some embodiments, module 160 may be used in connection with treating a single individual with individualized needs. In some embodiments, module 160 may be used to manufacture drug products for a group of individuals affected by the same ailment (for example, a localized viral outbreak or viral strain).

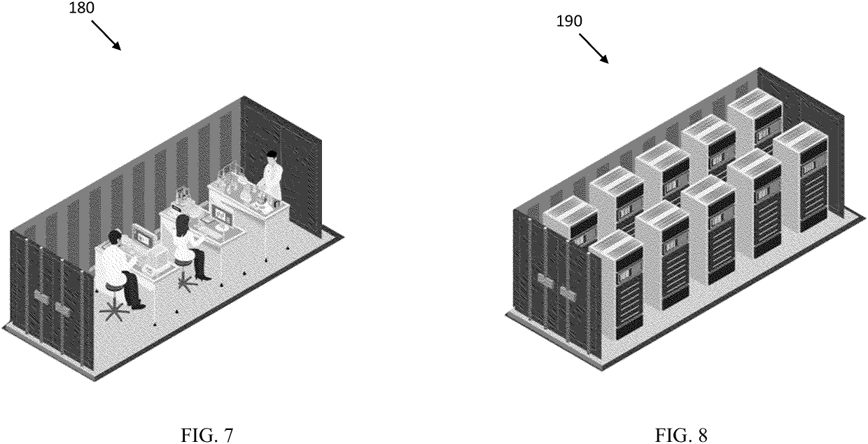

FIG. 7 depicts an overview of an exemplary drug product quality control site and/or module 180, according to aspects of the present disclosure. In some embodiments, a quality control assessment involves an assessment of presence of air and/or of one or more manifestations (e.g., loss of polydispersity, disruption of nanoparticle structure and/or of colloidal structure of an LNP composition, etc.) of air having been present.

FIG. 8 depicts an overview of an exemplary drug product warehouse site and/or module 190, according to aspects of the present disclosure.

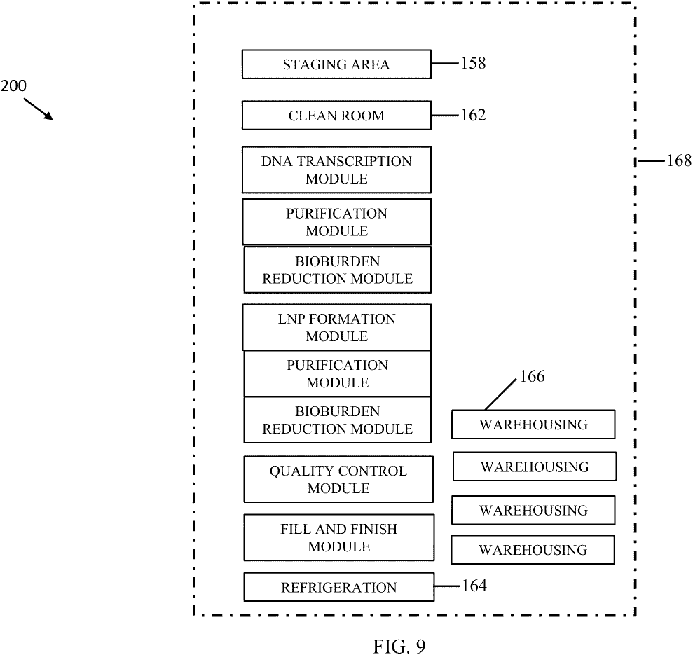

FIG. 9 depicts an overview of an exemplary drug product manufacturing site 200, according to aspects of the present disclosure. In the embodiment of FIG. 9, the site 200 may include about 15 modules or containers 102. In some embodiments, the site 200 may include from about 10 modules to about 20 modules or containers 102. The site 200 may include various numbers of each type of container 102 other than what is shown in FIG. 9. The site 200 may also include other types of modules or containers 102 (for example, HVAC modules), and in some embodiments may not include each and every type of module or container 102 illustrated in FIG. 9. The site 200 may include each of the modules or containers 102 shown in FIG. 3, as well as one or more staging area containers 158 (for example, for receiving shipments and/or visitors to the site 200). The site 200 may also include a clean room module 162 to allow personnel to change into clean working gowns and/or clothing. The site 200 may include one or more refrigeration modules 164 for freezing drug products as well as one or more warehousing modules 166 for storing drug products. The site 200 may be contained within a site boundary 168 that may include, for example, a giant tent, a hangar (for example, an inflatable hangar), and/or other types of temporary or permament structures, fences, warehouses and/or buildings.

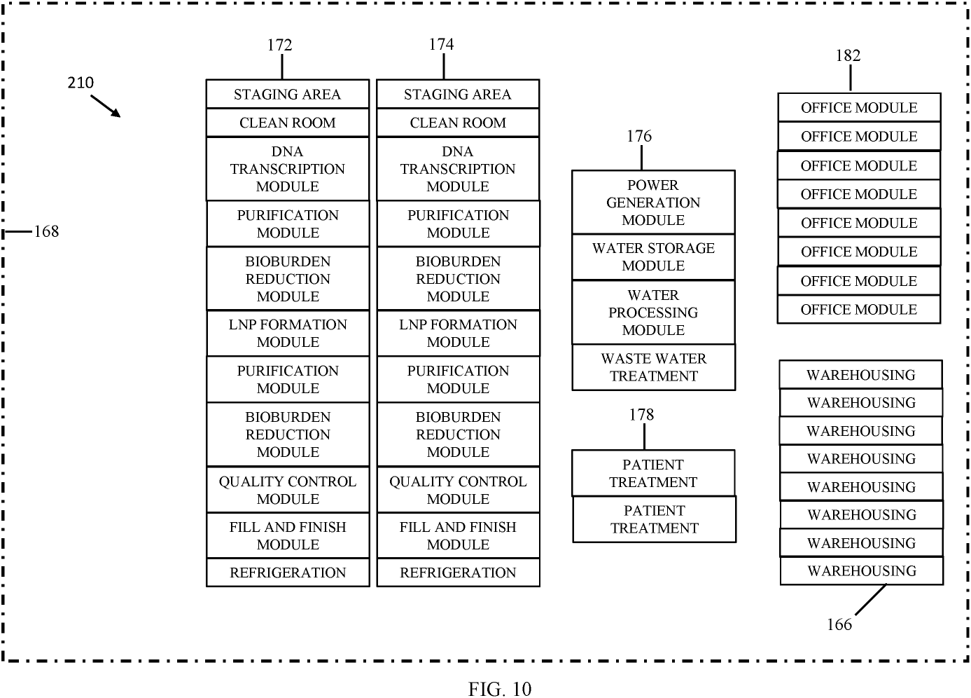

FIG. 10 depicts an overview of an exemplary drug product manufacturing site 210, according to aspects of the present disclosure. In the embodiment of FIG. 10, the site 210 may include about 40 modules or containers 102. In some embodiments, the site 210 may include from about 30 modules to about 50 modules or containers 102, or from about 10 to about 60 modules, and/or more than 60 modules. The site 210 may include various numbers of each type of container 102 other than what is shown in FIG. 10. The site 210 may also include other types of modules or containers 102 (for example, HVAC modules), and in some embodiments may not include each and every type of module or container 102 illustrated in FIG. 10. The site 210 may include each of the modules or containers 102 shown in FIG. 9, as well as a first drug production facility 172 and a second drug production facility 174, both located within the site boundary 168. The first and second drug production facilities 172, 174 may be used to produce the same drug products and/or different products, depending on the need. The site 210 may include a number of utilities 176 including power generation, water storage, water processing, and/or waste water treatment. The site 210 may also include one or more patient treatment areas 178, office areas 182, and/or warehousing modules 166.

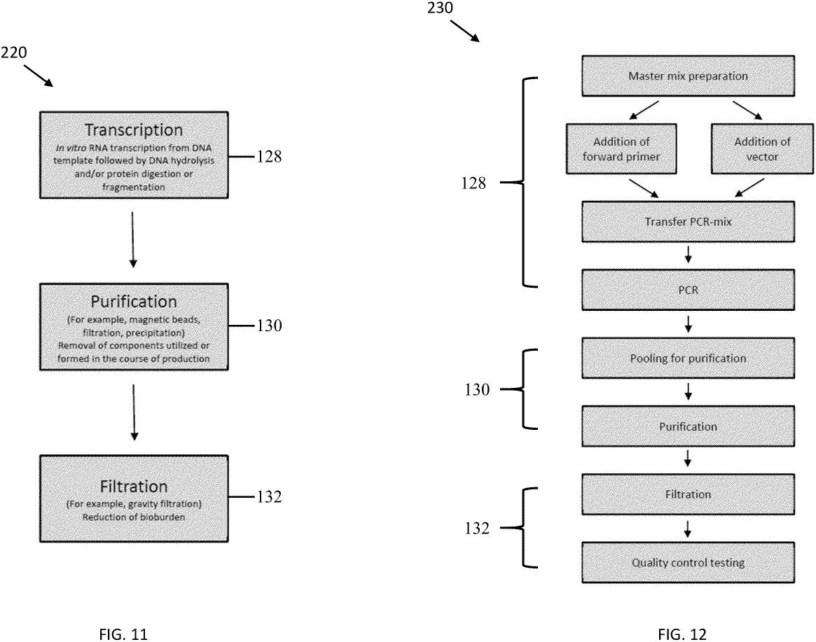

FIG. 11 illustrates an overview of exemplary manufacturing process 220 for a pharmaceutical-grade composition comprising RNA, according to aspects of the present disclosure. The process 220 may include the DNA transcription module 128, the first purification module 130, and the first bioburden reduction (or filtration) module 132, as previously described herein. In the embodiment of FIG. 11, the process 220 includes an exemplary manufacturing process for pharmaceutical-grade RNA comprising an in vitro RNA transcription followed by removal of components utilized or formed in the course of production by a purification process, and filtration to reduce bioburden (e.g., as illustrated in FIG. 11). Optional in-process controls may also be completed depending on whether a hold step is performed.

FIG. 12 illustrates an overview of exemplary DNA template manufacture process 230 via a PCR-based process, according to aspects of the present disclosure. In the embodiment of FIG. 12, the process 230 includes an exemplary manufacturing process of a DNA template via a PCR-based process including the DNA transcription module 128, the first purification module 130, and the first bioburden reduction (or filtration) module 132, as described herein. Initially, a master mix preparation is made. Subsequently, forward primer and vector were added. The PCR-mix is transferred into a reagent reservoir and a PCR plate was filled. A PCR is completed comprising an initial denaturation, a denaturation step, an annealing step, a final extension step for 20-30 (e.g., 20, 21, 22, 23, 24, 25, 26, 27, 28, 29, or 30) cycles and a hold step. The PCR products can be pooled and purified. Subsequently, the purified, pooled PCR product was filtered and quality control tested. FIG. 12 illustrates which portions and/or steps of the process 230 are contained within each module or container.

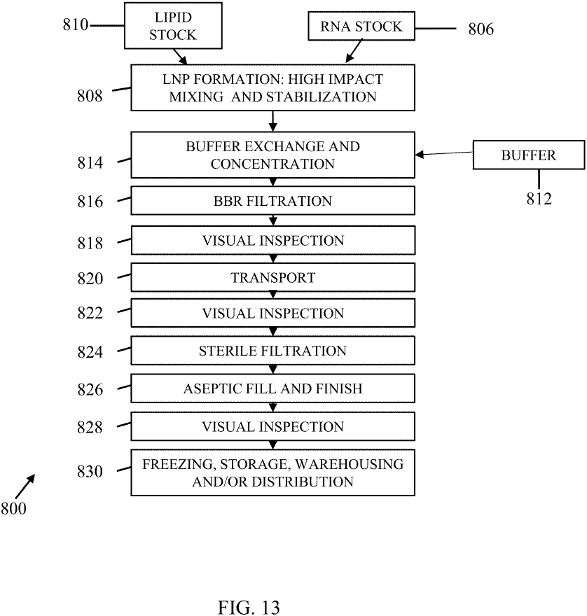

FIG. 13 illustrates an exemplary process 800 for manufacturing LNP compositions. Generally, steps 806, 808, and 810 (as well as equipment associated with those steps, as described herein) will occur and/or be located in the LNP formation module 134. Generally, steps 812 and 814 (as well as equipment associated with those steps, as described herein) will occur and/or be located in the second purification module 136. Generally, step 816 (as as well as equipment associated with step 816, as described herein) will occur and/or be located in the second bioburden reduction module 138. Steps following 816 may occur in other modules and/or may occur at other facilities (or not at all). For example, as explained herein, freezing and warehousing may not be required in all embodiments.

Referring still to FIG. 13, as can be seen, the produced compositions are prepared by combining lipids 810 with an aqueous preparation which carries an agent of interest (e.g., an active agent). In many embodiments, the agent of interest is a nucleic acid (e.g., a nucleic acid therapeutic). As depicted in FIG. 13, the nucleic acid is an RNA (e.g., a therapeutic RNA); in many embodiments of this depicted process, a utilized RNA includes at least one open reading frame (ORF) which may, for example, encode a vaccine antigen, a replacement protein, an antibody agent, a cytokine, etc). In some embodiments a vaccine antigen may be a cancer vaccine antigen or an infectious disease (e.g., viral) antigen. In some embodiments, an RNA encodes a polypeptide that is or comprises a viral antigen such as a coronaviral antigen, such as a spike protein or portion thereof, or relevant variant of the foregoing (e.g., a SARS-CoV-2 spike protein or receptor binding domain thereof, for example, a prefusion stabilized variant thereof), e.g., as is utilized in one or more of mRNA-BNT162a1, mRNA-BNT162b1, mRNA-BNT162b2, mRNA-BNT-162c1, mRNA-1273, CVnCov, CVnCoV2, etc.). In certain embodiments exemplified herein, utilized was an RNA of BNT162b2.

In some embodiments of the process depicted in FIG. 13, the RNA is prepared by in vitro transcription (e.g., of a DNA template which may, for example be a linear template such as a linearized plasmid or an amplicaon).

Among other things, as described herein, the present disclosure identifies the source of a problem associated with certain LNP compositions and/or their preparation, for example appreciating that presence of air can have undesirable impact(s). Without wishing to be bound by any particular theory, it is proposed that excess air, particularly in preparations or systems exposed to transport conditions, can adversely affect LNP compositions, for example resulting in aggregation or other loss of colloidal stability, and/or one or more other negative impacts of polydispersity. In some embodiments, the present disclosure provides an insight that such negative effects may be particularly likely and/or particularly deleterious in large scale preparations. Alternatively or additionally, the present disclosure provides an insight that such negative effects may be particularly problematic for preparations intended for filtration, e.g., before, during and/or after fill/finish steps such as are indicated in FIG. 13.

Referring to FIG. 13 and the exemplary process that it depicts, at step 808, the process 800 may include LNP formation by adding lipids 810 to an RNA solution 806, as well as high impact mixing (for example, via impingement jet mixing), and stabilization Typically, the RNA solution is an aqueous solution.

In many embodiments, the lipids 810 may include one or more of a cationically ionizable (sometimes referred to as “cationic” for simplicity) lipid, a phospholipid, a PEG-lipid, a sterol (e.g., a cholesterol) and an appropriate solvent (e.g., ethanol).

In some embodiments, LNP formation may be performed in presence of a buffer (e.g., a citrate buffer) 812. In some embodiments, the buffer (e.g., a citrate buffer) 812 may be present in the RNA solution 806 prior to mixing with the lipids 810 (for example, via in-line dilution of the water-diluted RNA with the buffer (e.g., citrate buffer) 812 to form the aqueous solution of RNA 806). Stated otherwise, buffer (e.g., citrate buffer) 812 may be added to the RNA solution prior to mixing with the lipid solution 810. In some embodiments, the buffer (e.g., citrate buffer) 812 may also or alternatively be added to the mixture resulting from combining the lipid solution with the aqueous solution 806 (which, as depicted in FIG. 13, is an RNA solution but could, in some embodiments, carry a different agent). In some embodiments, the buffer (e.g., citrate buffer) 812 may include citric acid (monohydrate sodium citrate) and/or sodium hydroxide.

According to embodiments described herein, step 808 (LNP formation) includes introducing substantially no air into the process and/or various solutions thereof, thereby forming a first RNA-LNP preparation that includes LNP-encapsulated RNA. LNP formation 808 may include the adjusting of one or more process temperatures, process flow rates, and/or ratios of the buffers, solutions and/or suspensions. LNP formation may include independently flowing each of the aqueous solution and lipids 810 (for example, in a lipid solution) into a mixing unit. Each of the aqueous RNA solution 806 and lipid solution 810 may flow into the mixing unit under laminar flow conditions (to avoid the entrapment of air bubbles within the flow).

Still referring to FIG. 13, at step 814, the process 800 may include buffer exchange and concentration of the first RNA-LNP preparation to form a second RNA-LNP preparation. The buffer exchange and concentration step 814 may be conducted with process parameters including, for example, a feed flow rate, for example within a range of of 18 to 50 liter/min (LPM), a trans-membrane pressure (TMP), for example lower than 1200 mbar, a retentate pressure, for example within a range of 130 to 230 mbar, and a permeate pressure, for example within a range of 10 to 70 mbar.

In some embodiments, buffer exchange 814 of the first RNA-LNP preparation and concentrating the first RNA-LNP preparation are performed in alternating steps. In one or more embodiments, a TRIS (i.e., tris(hydroxymethyl)aminomethane) buffer may be used. In some embodiments, the buffer exchange 814 is conducted via diafiltration and the concentration is conducted via ultrafiltration. In some embodiments, the diafiltration and/or the ultrafiltration are conducted via tangential flow filtration (TFF) (for example, in a tangential flow filtration unit and/or TFF skid). In some embodiments, the tangential flow filtration is conducted using one or more jejunostomy tubes and/or one or more dip tubes configured to avoid introducing air into the second RNA-LNP preparation. During the tangential flow filtration, a retentate may be recirculated to a feed tank using a dip tube comprising a first end submerged into filtration feed liquid in the feed tank to avoid introducing air into the filtration feed liquid. Prior to the buffer exchange and concentration steps, a filtration system for tangential flow filtration may be filled with a buffer to prevent introducing air into the second RNA-LNP preparation.

Referring still to FIG. 13, the buffer exchange and concentration step 814 may include at least two buffer exchanges conducted via diafiltration alternating with at least two concentrations conducted via ultrafiltration. During buffer exchange and concentration 814, process temperatures may be maintained within a desired temperature range (for example, at or below about 25° C., or from about 2° C. to about 25° C., or from about 15° C. to about 25° C.). During buffer exchange and concentration 814, pH may be continuously monitored (and may be maintained in a target range (for example, from about 7.0 to about 7.5, or from about 7.1 to about 7.3)) and shear may be maintained, for example in a range from about 2000 s^-1 to about 6000 s^-1, or from about 3000 s^-1 to about 5000 s^-1, or at about 4000 s^-1 (+/- 1%, 5%, and/or 10%). Following buffer exchange and concentration 814, a recovery flush may be performed, during which time shear may be reduced to under about 2000 s^-1 (for example, under about 1500 s^-1, or under about 1000 s^-1). In some embodiments, following buffer exchange 814, the pH may be maintained within a range from about 7.3 to about 7.5, for example following ultrafiltration and/or diafiltration.

In some embodiments, during buffer exchange and/or concentration 814, the pH of the first RNA-LNP preparation may be maintained at a pH that is higher than that of the cationic lipid (i.e., the cationic lipid in the lipid solution). Without wishing to be bound by any particular theory, it is proposed that doing so may reduce foaming of the liquid nanoparticles.

In some embodiments, the first and/or second RNA-LNP preparation(s) may be sterilized while introducing substantially no air into the produced formulation. In some embodiments, a relevant produced formulation may be a product for further manipulation, processing, packaging, and/or shipping. In some embodiments, a produced formulation may be or comprise a drug product formulation, e.g., for administration to humans.

In some embodiments, one or more sterilization steps may be performed by sterile filtration; in some embodiments, sterile (or other) filtration may be conducted at a target pressure with substantially no pressure building up during the filtration process, for example at about 1.03 bar (or from about 1.02 bar to about 1.04 bar, from about 1.01 bar to about 1.05 bar, or from about 1.00 bar to about 1.1 bar).

In some embodiments, a utilized mixing unit may include one or more impingement jet mixing skids. Prior to mixing, the impingement jet mixing skids may be vented and/or flooded to remove air from tubing of the impingement jet mixing skids. Mixing of the aqueous and lipid solutions may be performed within boundaries of the mixing unit and/or one or more impingement jet mixing skids. In some embodiments, prior to mixing, the aqueous solution does not contact the lipid solution. In some embodiments, the flow rate ratio into the mixing unit of the aqueous solution to the lipid solution is about 3:1. In some embodiments, the mixing speed may be adapted to avoid entrapping air in the first RNA-LNP preparation. For example, in order to avoid the introduction of air (and/or other impurities), one or more mixing processes may include increasing the mixing speed gradually until a slight vortex has formed (for example, the mixing speed at or slightly above the point at which a visible vortex has formed), but below the mixing speed at which foam begins to form.

Still referring to FIG. 13, the system (for example, the impingement jet mixing skids, the TFF system (i.e., the tangential flow filtration unit), and/or components thereof) may be assessed at one or more time points (e.g., monitored over time, e.g., periodically or continuously) for presence of air bubbles. In the event that air is detected in the aqueous solution, the lipid solution, the first RNA-LNP preparation, the second RNA-LNP preparation, the mixing unit, and/or tubing providing the aqueous solution of RNA, the lipid solution, the first RNA-LNP preparation, and/or the second RNA-LNP preparation, an alert or notification may be sent indicating that air has been detected somewhere in the system. In some embodiments, air detection may be performed via one or more flowmeters (for example, via one or more Coriolis flowmeters), and/or by visual assessment (e.g., via the human eye and/or various types of cameras), viand/or other detection means.

In some embodiments, the aqueous solution and/or the lipid solution may be flowed into the mixing unit through one or more inlets disposed at a bottom portion of the mixing unit, and the resulting first RNA-LNP preparation may be released from the mixing unit through one or more outlets disposed at a top portion of the mixing unit. In some embodiments, the mixing may be performed with a submerged mixer. In some embodiments, foam may be generated during and/or after formation of the LNP-encapsulated RNA, and may be subsequently removed from the RNA-LNP preparation (for example, the foam may be removed from the first and/or second RNA-LNP preparation).

Referring still to FIG. 13, following buffer exchange 814 and concentration, the process 800 may include 0.2 µm filtration and/or the addition of sucrose and PBS for compounding. Following compounding, the process 800 may include bioburden reduction filtration (BBR) 816 following the buffer exchange and concentration 814. Bioburden reduction filtration 816 may include filtering with 0.2 µm pore size (or for example, about a 0.22 µm pore size) or smaller filter. Bioburden reduction filtration 816 may also include using other pore sizes (for example, 0.45 µm pore size) as described herein. According to the present embodiments, 0.2 µm pore size filtering may also occur on each of the lipid solution and the aqueous RNA solution prior to mixing, on the first RNA-LNP preparation, and/or on the second RNA-LNP preparation. Bioburden reduction filtration 816 may also include filtering the post TFF-LNP suspension through a particulate reduction filter prior to filtering the suspension through (for example) the 0.2 µm pore size and/or 0.22 µm pore size bioburden reduction filter. In some embodiments, bioburden reduction filtration 816 may also include performing a filter recovery flush.

Still referring to FIG. 13, following bioburden reduction filtration 816, the process 800 may include filling transport bags (for example, Flexsafe ® bags) with the filtered second RNA-LNP preparation, and performing a visual inspection 818 of the transport bags for air bubbles. In some embodiments, transport bags may be, for example 12 L bags, 50 L bags, 100 L bags, and/or other suitable bag sizes (e.g., depending on the batch size of the relevant RNA-LNP preparation), including bags that include a volume between 12 L and 50 L, and/or bags that include a volume between 50 L and 100 L. As noted herein, the present disclosure appreciates that negative impact(s) of introduced air may be particularly problematic when LNP compositions (e.g., RNA-LNP preparations as exemplified herein) are manufactured on relatively large scale and/or need to be transported.

In some embodiments, filling transport bags may include filling the bags to a volume in a range from about 30% to about 95%, or from about 40% to about 90%, or from about 50% to about 85%, or from about 60% to about 85% or from about 70% to about 85%, and/or other subranges therebetween of the total bag volume. In some embodiments, prior to filling, the bags may be evacuated, unfilled, and/or otherwise uninflated, in order to avoid the introduction of air bubbles.

In some embodiments, after filling the bags to the desired volume, air bubbles may be removed (e.g., may be manually removed from the bags via syringe), and the bags may be sealed. In some embodiments, care is taken to ensure no air bubbles are or become entrapped therewithin during sealing. After the bags are filled and sealed, visual inspection 818 may be performed and may include visual inspection 818 using the human eye, and/or camera. In some embodiments, if air bubbles are discovered in bags, efforts may be made to remove the air bubbles (e.g., manually via syringe); alternatively or additionally, in some embodiments, bags with bubbles (e.g., bags with visibly observable bubbles) may be discarded.

Filled bags may be stored and/or shipped at a temperature in a range from about 1 degree C to about 15° C. (for example, at about 2° C. to about 10° C., or from about 2° C. to about 8° C.), or alternatively may be frozen to a temperature of about -70° C. (for example, in a range from about -60° C. to about -80° C.). Prior to shipment, (e.g., once any air bubbles have been removed), the bags may be secured in or on racks and/or within or on any other suitable shelving or storage system so as to minimize movement, rupturing, and/or disruption of the bags during the transport to a fill and finish site. For example, transport bags may be stacked in a specific manner using a stacking system on pallets that include shock absorbers. During transport 820 and/or in preparation for transport 820, as well as following transport, nitrogen with a positive pressure (for example, from about 1-2 bars) may be maintained in and around the environment in which the bags are kept and/or transported, in order to prevent air from entering the bags. After transport 820, the bags may be assessed for air content (e.g., visually inspected) 822 a second time. In some embodiments, air bubbles that are discovered during such second assessment 822 may be removed (e.g., may be manually removed), or alternatively, the bag or bags that include air bubbles may be selectively discarded (for example, if the volume of air within a given bag has exceeded a threshold).

Referring still to FIG. 13, after arriving at a fill and finish site (and, in some embodiments, after having been assessed for air a second time), sterile filtration 824 may be performed (i.e., the second RNA-LNP preparation). In some embodiments, such sterile filtration 824 may be performed after the preparation has been removed from the transport bags, but prior to being disposed within a collection vessel, reservoir, and/or fill tank. In some embodiments, the material (i.e., the filtered preparation) may then be dispersed from the collection vessel, reservoir, and/or fill tank during aseptic fill and finish 826 (for example, to aseptically fill glass vessels with the drug product).

A third air assessment (e.g., by visual inspection) 828 may be performed on the filled glass vessels, again to ensure no air bubbles have been introduced. The inspected and filled glass vessels, at step 830 of the process 800, may then be frozen, stored, warehoused and/or distributed, for example, to health care administration sites. Alternatively, in some embodiments, filled glass vessels may be subjected to lyophilization or other drying process, so that drug product is transported and/or stored in a dry state (e.g., for subsequent resuspension).

In many embodiments, aseptic fill and finish 826 at the fill and finish site as depicted in FIG. 13 will be performed such that substantially no air is introduced to the product.

Still referring to FIG. 13, in some embodiments, the fill and finish facility may be located in the same location as the LNP production facility, in which case fill and finish may be performed directly using Point of Fill filtration equipment (in which case the transport 820, bag filling and sealing, and one or more of the visual inspection steps 818, 822, 828 may not be required. In yet other embodiments, the process 800 may include multiple transport steps 820, as well as additional visual inspection steps 818, 822, 828 if the various steps of the process 800 are performed at additional and/or other facilities (or alternatively, if transport is required within a single facility).

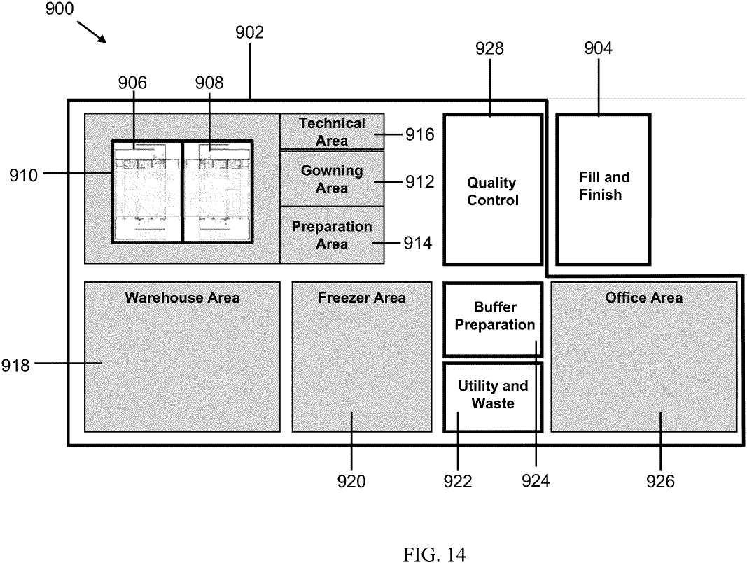

FIG. 14 depicts an overview of an exemplary drug product manufacturing site 900, according to aspects of the present disclosure. In the embodiment of FIG. 14, the site 900 may include a site boundary 902 defining the various areas located at the site 900. In some embodiments, the fill and finish process 904 may be performed at a different location (or may occur on site 900). The site 900 may encompass a total secured ground area of 6,000 m² to 8,000 m², or from about 5,000 m² to about 10,000 m², with a total of about 3,000 m² to 6,000 m², or from about 4,000 m² to about 5,000 m² total sheltered area (that is, total building area). The site 900 may include a drug substance module 906 and a drug product module 908, both enclosed within a clean area boundary 910. Each of the drug substance and product modules 906, 908 may be arranged in a 6-container configuration similar to the arrangements shown in FIG. 4 (i.e., similar to drug substance module 124 and drug production module 126). The drug substance module 906 and the drug product module 908 may encompass a total area of about 800 m², or from about 500 m² to about 1,000 m². Adjacent to the drug substance module 906 and the drug product module 908, the site may include a gowning area 912 and a preparation area 914, which in some embodiments may encompass a combined area of about 300 m². A technical area 916 for electronics, utilities, facility control equipment, as well as other equipment may be located adjacent the gowning area 912.

Referring still to FIG. 14, the site 900 may include a warehouse area 918, a freezer area 920, a buffer preparation area 924, a utility and waste area 922, an office area 926, and a quality control area 928. The warehouse area 918 may encompass a total area of of about 600 m², or from about 500 m² to about 800 m², and may be configured to include inbound and/or outbound logistics to accommodate 1,000 palette positions (for example, each with a standard pallet size of about 1000 mm × 1200 mm or about 800 mm ×1200 mm). The freezer area 920 may include at least three (3) climate zones, with a first zone maintaining temperatures at or around -70° C. (for example, from about -60° C. to about -80° C.), with a second zone maintaining temperatures at or around -20° C. (for example, from about -10° C. to about -30° C.), and with a third zone maintaining temperatures in a range from about about 2° C. to about 8° C.). In some embodiments, each of the buffer preparation area 924, the utility and waste area 922, the office area 926, the quality control area 928, and/or the fill and finish area 904 may be provided (and/or initially constructed) by the local country and/or region where the site is located, rather than being shipped to the site in one or more standard shipping containers. In some embodiments, the quality control area 928 may encompass an area of about 400 m², the buffer preparation area 924 may encompass an area of about 200 m², the utilities and waste area 922 may encompass an area of about 200 m², the fill and finish area 904 may encompass an area of about 400 m², and the office area 926 may encompass an area of about 800 m². The site 900 may also include a security gate / guard house and/or other suitable structures. In some embodiments, the site may include a second drug product module 908, which would serve to increase the production capacity (for example, to a number greater than 50 million doses per year such as about 80-100 million doses per year), and may add about 300 m² to the site footprint.

Still referring to FIG. 14, the drug substance module (or area) 906 and the drug production module (or area) 908 may be housed in a building with a ceiling height of at least 8 m, with a point load capacity of at least 9,000 kg, and with gates and/or doors with a width of at least 4 m and a height of at least 7 m. The warehouse area 918 (as well as other areas such as the quality control area 928, the buffer preparation area 924, the utility and waste area 922, the fill and finish area 904, the drug substance module 906 and the drug product module 908) may be maintained at a controlled humity and at a temperature from about 15° C. to about 25° C. The freezer area 920 may include space for about 10-20 freezers as well as about 5 to about 10 refrigerators. The utility and waste area 922 may provide a normal power supply in a range of about 2,000 kW to about 4,000 kW (for example, about 3,000 kW), and may include an uninterrupted power supply of at least 250 kW (for example, from about 200 kW to about 300 kW), to supply power for the entire site. The drug substance and drug product modules 906, 908 may require a total (i.e., combined) normal power of about 300 kW (for example, in a range from about 200 kW to about 400 kw) with an uninterrued power requirement of about 70 kW (for example, in a range from about 60 kW to about 80 kW, or form about 50 kW to about 100 kW). The utility and waste area 922 may also include a drinking water supply capable of delivering a volume of 4 m³/hr of drinking water to the site. The utility and waste area 922 may also include provisions for providing network connections to the site 900, as well as compressed air, nitrogen, carbon dioxide, and/or oxygen (for example, supplied in bottles). The site 900 may also include various other components, layouts, and arrangements of modules other than what is shown in FIG. 14.

Referring still to FIG. 14, the fill and finish module 904 (or area) may include the necessary equipment for carrying out the following process steps: pooling of liquids or frozen bulk material (for example in grade C conditions); sterile filtration into one or more asceptic filling lines under grade A conditions (for example, via Isolator Technology and/or RABS); capping and crimping of (for example) 2R ISO 8362-1 standard vials up to (for example) 2.25 mL; performing manual, semi-automated, and/or fully automated visual inspection of the filled vials; serialization and labelling of the vials; freezing and storage of the filled vials; and packing and shipping under dry ice. The fill and finish module 904 (or area) may include the necessary equipment to handle about 10 million vials (the equivalent of 50 million doses) annually. In preferred embodiments, the fill and finish module 904 meets one or more standards (for example, the cGMP standard and/or Process Control systems such as Siemens PCS7). In some embodiments, the fill and finish area 904 is located on site 900, while in some embodiments, the fill and finish area 904 is located off site.

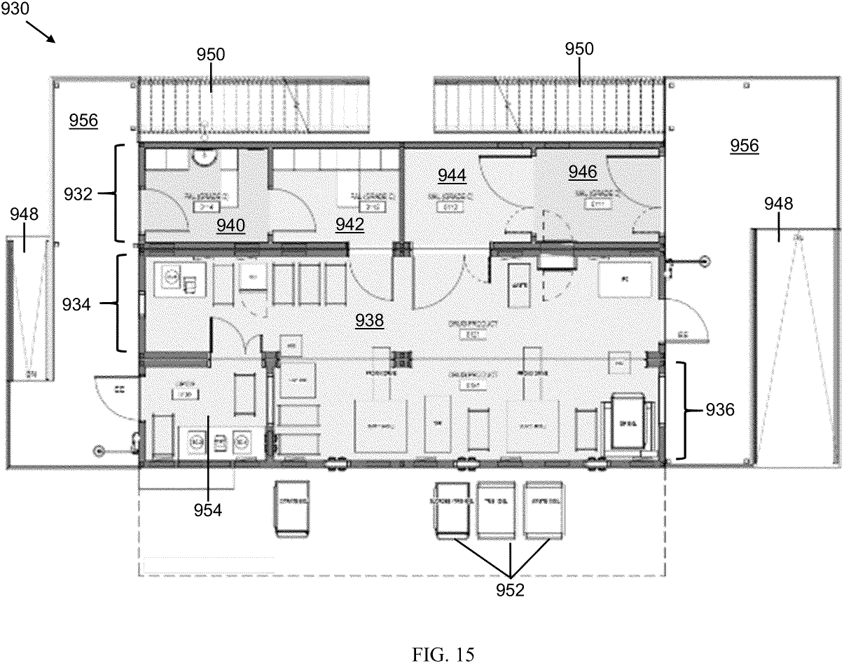

FIG. 15 depicts an overview of an exemplary drug substance module 906 and/or drug product module 908, according to aspects of the present disclosure. The illustration of FIG. 15 shows a possible layout that could be used for both the drug substance module 906 as well as the drug product module 908. The drug substance module 930 and/or drug product module 930 may include 3 containers 932, 934, 936 on a first level (for example an upper level or lower level) of a production facility. The embodiment of FIG. 15 is similar in layout to the 6-containeer drug substance module 124 and/or drug product module 126 shown in FIG. 4. As such, FIG. 15 may include an additional 3 containers housing HVAC equipment (for example, on a level below or above the level shown in FIG. 15. The first container 932 may be used primarily for staging and for ensuring an airlock is maintained, while the second and third containers may be used primarily to house the operation suite (i.e., for manufacturing drug substance and/or drug product). In a first end, the first container 932 may house a personnel entry section 940 adjacent a personnel airlock area 942. Within a second end, the first container 932 may house a material entry area 946 adjacent to a material airlock area 944. Upon entry within the first container 932, personnel and/or materials must travel through the respective personnel and/or material airlock sections 942, 944, in order for ambient and/or environmental air to be flushed out (for example, via vacuum pumps and/or blowers in cooperation with sealed doors and entryways) to ensure no impurities enter the operational suite 938 when personnel and/or materials enter. In the course of normal operations, personnel and materials should only enter the operation suite 938 through the respective airlocks. The drug substance module 906 and/or drug product module 908 may include manufacturing and/or production equipment within the operation suite 938, as described in the present disclosure. The location of equipment, walls, doors, and/or other structures may be adjusted from what is shown in FIG. 15 as needed to optimize a given production process.

Referring still to FIG. 15, the drug substance module 906 and/or drug product module 908 may include an APEX or hazardous equipment area 954 for housing any materials and/or equipment that is electrified, pressurized, heated and/or otherwise presents a risk of fire, combustion, and/or explosions (for example, equipment falling within the scope of the ATEX 114 “equipment” Directive 2014/34/EU and/or the ATEX 137 “workplace” Directive 1999/92/EC). The hazardous equipment area 954 may also be maintained as a clean room to reduce the likelihood that combustible material and/or debris will enter the hazardous equipment area 954. In some embodiments, the hazardous equipment area 954 (or “ATEX room”) may be used to house one or more drug product components or subcomponents used for lipid preparation, which may require the use of ethanol, which may (along with disinfectants) be stored in one or more safety cabinets within the ATEX room 954. The drug substance module 906 and/or drug product module 908 may also include one or more stairways 950 (for example, leading up or down to the HVAC level) as well as one or more ramps 948 to facilitate the ease with which materials (and/or personnel) may be transported to one or more platforms 956 leading to the personnel and material entryways 940, 946. The drug substance module 906 and/or drug product module 908 may include additional external equipment 952 such as transformers, tanks, HVAC equipment, and other necessary components located outside of the containers 932, 934, 936, but in fluid, electrical, thermal, and/or operative communication with components in the interior of the containers 932, 934, 936.

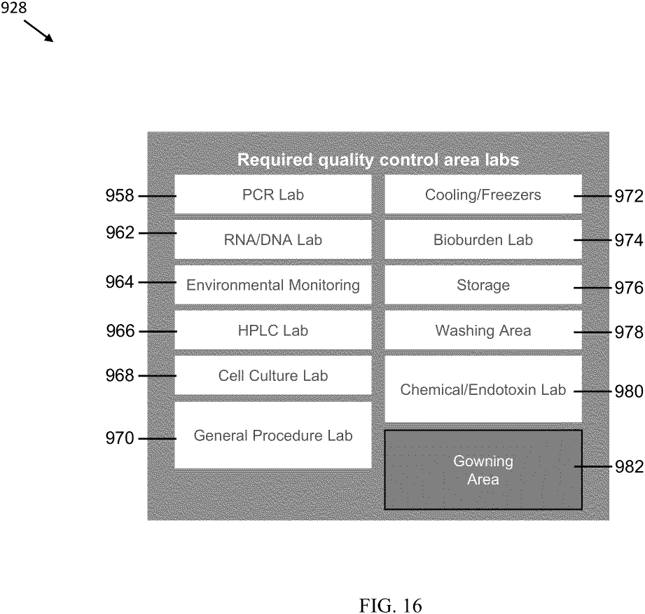

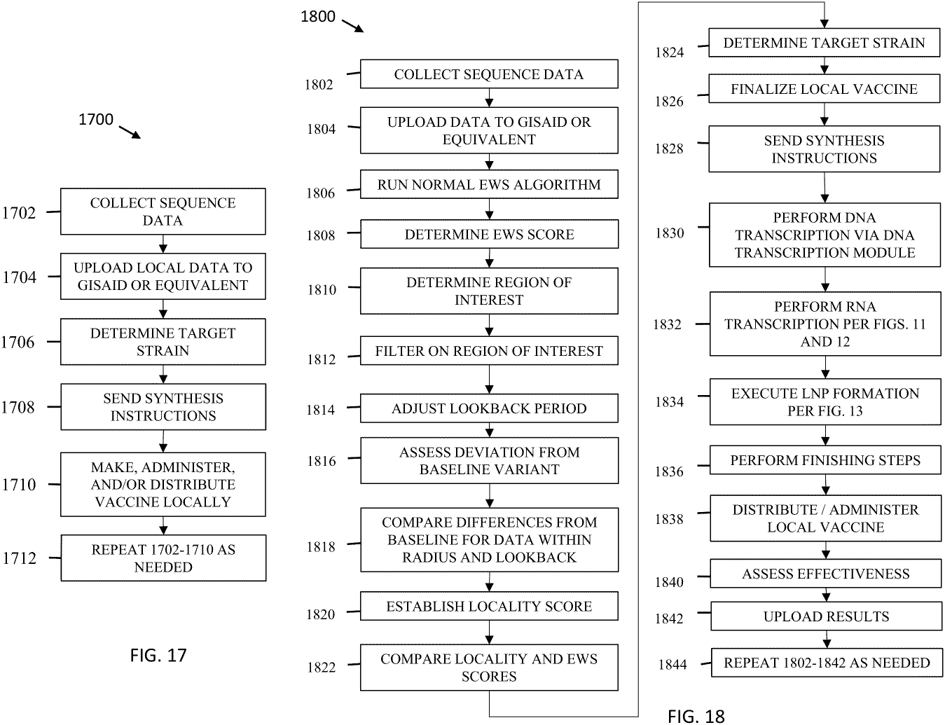

FIG. 16 depicts an overview of an exemplary quality control module 928, according to aspects of the present disclosure. The quality control module 928 may include a PCR lab 958, an RNA/DNA lab 962, an environmental monitoring console 964, a high performance liquid chromatography (HPLC) lab 966, a cell culture lab 968, a general procedure lab 970 (for example, for monitoring such parameters as pH, color, sample logistics, and/or storage metrics), freezer monitoring equipment 972, a bioburden lab 974, a quality control storage area 976, a washing area 978, a chemical/endotoxin lab 980, and/or a gowning area 982. The processes by which quality is controlled (for example, within each of the processes and/or labs illustrated in FIG. 16) may be tightly controlled and/or standardized to reduce the introduction of sources of variation into each lab. The quality control module 928 may be configured such that access by staff/personnel to each of the lab areas is possible only through the gowning area to ensure proper sanitizing and disinfecting measures are taken prior to entering the lab areas within the quality control module 928.

In connection with the modular drug production system 100 of the present embodiments, various personnel and staffing needs must be met in order to ensure consistent and satisfactory operations of the modular drug production system 100. For example, in connection with the drug substance module 906 and/or the drug product module 908, qualified operators / personnel are needed for: operating bioreactors and ensuring laminar flow, monitoring purification protocols, monitoring formulation and filtration processes, process support protocols, as well as production and/or automation engineers, scientists, and/or technicians. In connection with the quality control module 928, scientists and/or technicians with laboratory experience are required for performing various tests to ensure quality control is maintained. In connection with the fill and finish module 904, staff is needed for filling operations, optical control, labelling, and packing. In connection with the warehouse area 918, personnel are needed for logistics and warehousing, while the office area 928 requires personnel with a wide range of skills including (but not limited to) subject matter experts, scientists, engineers, automation experts, quality control / quality assurance experts, procurement / supply chain specialists, finance professionals, human resource professionals, training managers, IT administrators, and overall operations coordinators.

The modular drug production system 100 of the present embodiments may be transported to (and further built or developed as needed) at sites that have solid street access, access via train, and/or shipping to allow each of the containers and other equipment and materials to be transported to site. In some embodiments, the modular drug production system 100 may be transported to (and further built at) existing pharmaceutical installations such that existing laboratory equipment (for example, to be used as a quality control module 928) and/or fill and finish capacity may be utilized. Proximity to universities may also be beneficial to allow staffing needs (for example, including employees / personnel with the requisite technical backgrounds) to more easily be met. In preferred embodiments, sites are located within 1 hour from airports, harbors, and/or train stations to help facilitate logistics and supply chain operations. In some embodiments, the site may be situated in more remote locations, in which cases additional time should be allowed for materials and equipment to arrive at sight, and careful consideration should be taken to ensure that the required qualified personnel will be available to help operate the modular drug production system 100. In addition, and as discussed in this disclosure, the site or location of the modular drug production system 100 installation requires access to power, drinking water, internet/network connections, and must be able to be continuously secured (that is, 24 hours a day, seven days a weeks).

The modular drug production system 100 of the present embodiments may be used in connection with 12.8 gram, 25.6 gram, 38.4 gram, 40 gram and/or other sized production scales. For example, the modular drug production system 100 of the present embodiments may be used in connection with drug product outputs that include 25 L bags of 12-gram batches. Various numbers of containers and types of containers have been described in connection with the embodiments of the present disclosure. It should be appreciated that the modular drug production system 100 of the present embodiments may be used in connection with other types and numbers of containers than those explicitly described here. Standard shipping containers, as described herein, may include a width of about 8 ft. (2.43 m), a height of about 8.5 ft. (2.59 m) and a length from about 20 ft. (6.06 m) to about 40 ft. (12.12 m). Other standard sizes and half sizes and/or large and small sizes may also be used. The modular drug production system 100 of the present embodiments may be used in connection with processes of various production scales. As such, some of the process parameters may vary, while much of the general process flow and equipment will largely remain the same for micro, small, medium, and large scale drug production. The modular drug production system 100 of the present embodiments has been generally described in connection with RNA-LNP drug production, but may also be used in connection with the production of other drugs.

### Early Warning System

In one or more aspects, the present embodiments are directed to a localized Early Warning System (EWS) for identifying disease Variants of Concern (VOC). In one or more aspects, the present embodiments are directed to systems and method for determining and producing vaccines that target and help to treat local strains of a disease. In some embodiments, Early Warning Systems are used to identify Variants of Concern (i.e., in the context of worldwide spread) based on one or more of: the transmissibility of a strain, the rate of growth of a strain within a human being, the ability of strain to evade existing vaccines and boosters, and the severity of the strain to humans. (See: Early Computational Detection of Potential High Risk SARS-CoV-2 Variants; Beguir et al, (doi: https://doi.org/10.1101/2021.12.24.474095)).

FIG. 17 illustrates a process (or method) 1700 for making vacciens, according to aspects of the present disclosure. At step 1702, the method 1700 may include collecting sequence data (for example, using local sequencing equipment based on test samples taken from a local area). At step 1704, the method 1700 may include uploading the local data to a public database such as GISAID, NCBI, an EWS database, and/or other databases. At step 1706, the method 1700 may include analyzing the data (i.e., data local to the area of interest) to determine a target strain. The target strain may be an actual strain that is prevalent in the area and/or may be a hybrid that combines different segments of sequences of interest to target a hybrid or “synthetic” strain that is representative of one or more local strains. At step 1708, the method 1700 may include sending synthesis instructions from the database and/or cloud-basd computing system to a local computing system, the synthesis instructions including sequencing information such that DNA can be created via DNA synthesis. At step 1710, the method 1700 may include making, administering, and/or distributing a vaccine locally, as described herein, the vaccine being based on the synthesis instructions. At step 1712, the method 1700 may include repeating steps 1702-1710 as needed.

FIG. 18 illustrates a process (or method) 1800 for making vaccines, according to aspects of the present disclosure. At step 1802, the method 1800 may include collecting sequence data. At step 1804, the method 1800 may include uploading the local data to a public database such as GISAID, NCBI, an EWS database, and/or other databases. At step 1806, the method 1800 may include running a conventional EWS algorithm on the collected data to identify and assess potential Variants of Concern based on one or more of the transmissibility of a strain, the rate of growth of a strain within a human, the ability of strain to evade existing vaccines and/or boosters, and the severity of the strain to humans. In some embodiments, running the EWS algorithm includes a lookback period of 1-week (i.e., evaluating data collected in the most recent week). At step 1808, the method 1800 may include determining an EWS score (i.e., quantifying and assigning a score to each evaluated strain in order to determine if a Variant of Concern has been identified for further study, and on which potential future action may be taken). At step 1810, the method 1800 may include determining a region of interest (for example, the local area in which the site is located and/or potential adjacent or proximal areas within which particular outbreak activity has been occurring). In some embodiments, a region of interest may include a simple radius around a geographic area (for example, within 50 miles, 100 miles, 500 miles, 1000 miles, etc. of the site). In some embodiments, a region of interest may include certain population centers that do not fall neatly within a certain radius (since population density is rarely uniform, and transport between regions is also typically non-uniform (and well-traveled routes may be conduits for disease spread)).

Referring still to FIG. 18, at step 1812, the method 1800 may include filtering the data on the public database based on the region of interest (i.e., so as to include only sequence data from strains from within the region of interest). At step 1814, the method 1800 may include adjusting the lookback period (for example, filtering the data based on a particular time period of interest). Adjusting the lookback period may include looking back more than a single week (for example, looking back a month, or several weeks, etc. as may be the case). Adjusting the lookback period may include selecting a range of dates that coincides with the dates that correspond to local outbreaks, etc. At step 1816, the method 1800 may include assessing deviations from the filtered data and one or more baseline variants. Assessing deviations may include creating a deviation between each sequence within the filtered data set and a baseline variant. Assessing deviations may also include creating a deviation between certain subsets of sequences within the filtered data set and a baseline variant. For example, sequences within the filtered data set may be put into one or more bins or subsets, and averaged such that an average or representative sequence may be determined for each subset. Deviations between each representative sequence and the baseline sequence may then be determined. In some embodiments, assessing deviations may include identifying regions of the representative sequence that deviate from the baseline variant. In some embodiments, assessing deviations may also include the specific mutation associated with each nucleotide that deviates from the baseline variant. In some embodiments, the baseline variant may include a previously identified strain that is or was prevalent within the region of interest. In some embodiments, the baseline variant may include a target strain upon which an existing vaccine is based.

Referring still to FIG. 18, at step 1818, the method 1800 may include comparing deviations from the baseline variant for each subset of data within the radius and lookback period (i.e., within the filtered data set) to determine if there are commonalities between the regions of the sequence where deviations occur and/or between the specific mutations for each of the deviations. At step 1820, the method 1800 may include establising locality scores for each subset within the filtered data subset, the localitiy scores being based on commonalities that each subset shares with other subsets within the filtered data (thereby identifying the most common strains that are specific to the location and time period of interest). At step 1822, the method 1800 may include comparing locality and EWS scores to assess and/or inform a future direction for Variants of Concern and/or local vaccine decision-making. For example, if the EWS scores are low, it means there likely have not been any Variants of Concern identified from a worldwide outbreak point of view. On the other hand, if the locality score is high, it means that the strains in the region of interest and in the time period of interest vary greatly from a baseline variant, meaning that existing vaccines may not be particularly well-suited for treating local strains within the region of interest, and/or the potential for future mutation is higher since the local strains have already deviated significantly from the baseline varant.

At step 1824, the method 1800 may include determining a target strain based on the EWS score, the locality score, and/or a combination thereof. At step 1826, the method 1800 may include finalizing one or more vaccines (i.e., based on the target strain for the given region of interest, the target strain being an actual strain and/or a hybrid or synthetic strain that prioritizes aspects of sequences resulting from both the EWS and the local strain analyses). At step 1828, the method 1800 may include sending synthesis instructions from the database (or network, or cloud, and/or command center) to the site. At step 1830, the method 1800 may include performing DNA synthesis based on the synthesis instructions using a DNA synthesizer located within the transcription module (and/or within a different module) according to the present disclosure. DNA synthesis may be followed by transcription, accoridng to aspects of the present embodiments, and as described herein. At step 1832, the method 1800 may include perfoming RNA transcription per FIGS. 11 and 12. At step 1834, the method 1800 may include performing LNP formation per FIG. 13. At step 1836, the method 1800 may include performing finishing steps (i.e., fill and finish) as described herein. At step 1838, the method 1800 may include distributing and/or administering the local vaccine within the region of interest. At step 1840, the method 1800 may include assessing the effectiveness of the local vaccine for treating the local strain of the disease. At step 1842, the method 1800 may include uploading the effectiveness results to the one or more public databases. At step 1844, the method 1800 may include repeating any of steps 1802-1842 as needed.

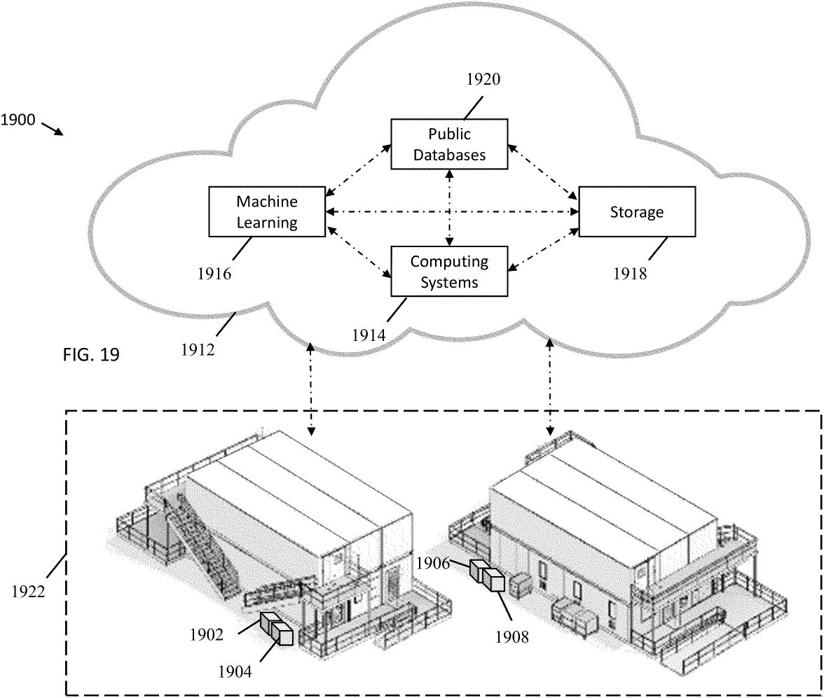

FIG. 19 depicts an overview of an exemplary drug product manufacturing enterprise and/or process, according to aspects of the present disclosure. In the embodiment of FIG. 19, certain aspects of the enterprise may be located or occur at the site 1922 or alternatively, on the cloud (or network) 1912. According to aspects of the present embodiments, the site 1922 may include one or more sequencers 1902 to produce sequences of local strain. The site 1922 may also include one or more DNA synthesizers 1906 for synthesizing DNA (upon which vaccines may be generated, according to the present embodiments), the DNA sysnthesis being based on target strains, as determined from the cloud or network 1912. The cloud or network, which is communicatively coupled to the site 1922 and able to both transmit data to, and receive data from, the site 1922, may include several functions and components including (but not limited to) machine learing 1916, public databases 1920, data storage 1918, and computing systems 1914.

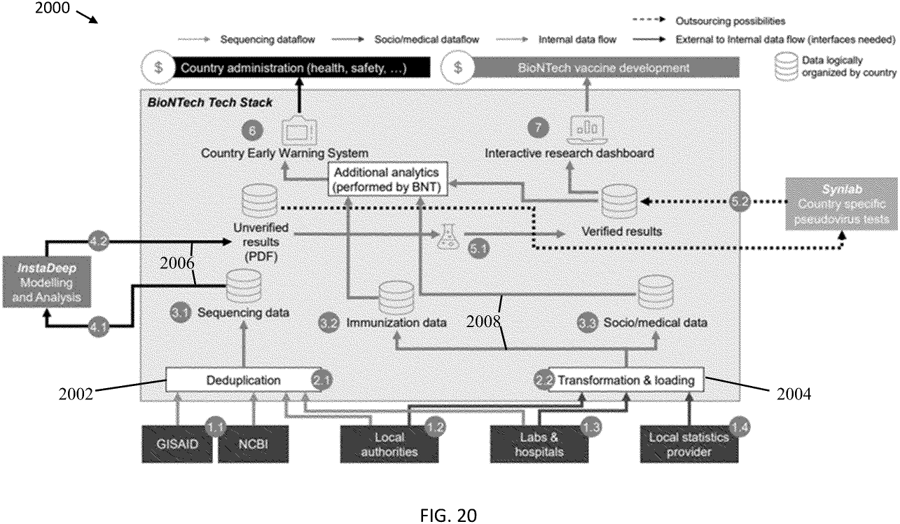

FIG. 20 depicts an overview of another exemplary drug product manufacturing enterprise and/or process 2000, according to aspects of the present disclosure. In the embodiment of FIG. 20, sequencing dataflows (i.e., to deduplication 2002), socioeconomic and medical dataflows (i.e., to transformation and loading 2004), external dataflows (i.e., to modelling and analysis), and internal data flows 2008 are illustrated, according to aspects of the present embodiments.

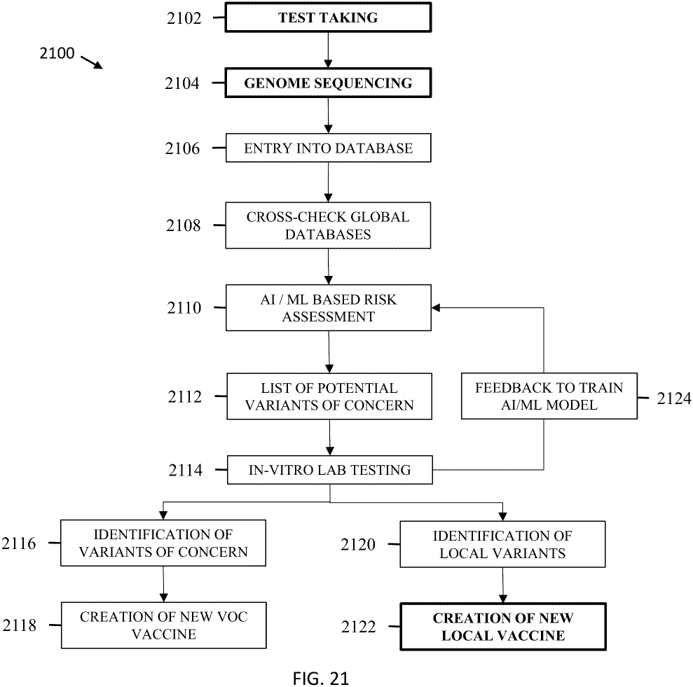

FIG. 21 depicts an overview of another exemplary drug product manufacturing enterprise and/or process (or method) 2100, according to aspects of the present disclosure. At step 2102, the method 2100 may include taking a test (for example, at the site). At step 2104, the method 2100 may include genome sequencing one or more samples from the test. At step 2106, the method 2100 may include entering genome sequencing data into a database. At step 2108, the method 2100 may include cross-checking the entered genome sequencing data against one or more global databases. In some embodiments, cross-checking the entered genome sequencing data against one or more global databases may include comparing the entered genome sequencing data against other local genome sequencing data and/or genome sequencing data from a global sequence repository. If the entered genome sequencing data is not in the repository, it may be added, according to aspects of the present embodiments. At step 2110, the method 2100 may include using machine learning or artificial intelligence to perform one or more risk assessments on the genome sequencing data. At step 2112, the method 2100 may include identifying a list of potential variants of concern based on the one or more AI-based risk assessments. At step 2114, the method 2100 may include performing in-vitro lab testing to verify and confirm the prediction(s) from the AI-based risk assessments. At step 2124, the method 2100 may include providing feedback from the in-vitro lab testing to the AI or machine learning model, in order further refine the AI or machine learning model.

Referring still to FIG. 21, at step 2116, the method 2100 may include identifying one or more variants of concern (VOC) following the confirmation and/or verification during in-vitro lab testing. At step 2118, the method 2100 may include creation of a new VOC vaccine to treat the one or more variants of concern (VOC). At step 2120, the method 2100 may include identifying one or more local variants following the confirmation and/or verification during in-vitro lab testing. At step 2122, the method 2100 may include creation of a new local variant vaccine to treat the one or more local variants. In some embodiments, the bolded steps shown in FIG. 21 (i.e., steps 2102, 2104, and 2122) may occur at the local site while the un-bolded steps (i.e., the remaining steps of FIG. 21) may occur at the enterprise and/or cloud level.

According to the present disclosed embodiments, variant and/or local strain sequencing data from surveillance field testing can be processed in real time (or near real time) without needing to ship samples to centralized testing locations, which could add days to the turnaround time. AI and machine learning running on a supercomputing infrastructure (for example, leveraging a cloud-based network) allows results and analysis to be made available on site. In some embodiments, the dislosed systems and methods rank all known variants for infectivity and immune escape risk potential, allowing a live monitoring of the worldwide pandemic and local outbreaks. Novel variant sequences can be scored and ranked in silico (i.e., via computer modelling) in mere hours (for example, less than 4 hours, less than 3 hours, less than 2 hours, etc.). The present embodiments allow new variants to be identified and added to the global pool or repository more quickly and thoroughly. Similarly, the present disclosure enables the most infectious variants, as well as the most immune escaping variants, to be detected. The present embodiments also provide a ranked list of variants that should be monitored and tested in-vitro, which helps to prioritize how laboratory resources may most effectively be utilitized.

The present embodiments provide the ability to produce mRNA drug products (for example, vaccines) at locations worldwide. By incorporating a distributed network of modular drug production installations into a worldwide Early Warning System (EWS), the present embodiments provide infrastructure and on-the-ground capabilities to help battle ongoing and future pandemics. The present disclosure provides systems and methodologies that allow greater access to vaccine and disease treatment, regardless of socioeconomic status and other factors. In addition, localized production facilities are well-suited to provide tailored and real-time (or near real-time) response to local outbreaks. The provided ecosystem also increases testing and research capacity and encourages collaboration. The EWS of the present embodiments enables potential identification of pathogens that pose a pandemic threat via AI-powered risk assessment and confirmatory lab testing. In addition, by having a worldwide network of on-the-ground installations, increased transparency of pandemic threats may be realized through faster detection of new variants and/or pathogens. The local modular mRNA vaccine production units of the present embodiments also reduce the dependency of states and countries on external drug supplies and suppliers, thereby ensuring rapid and local responses to potential future pandemic situations.

### EQUIVALENTS

Those skilled in the art will recognize, or be able to ascertain using no more than routine experimentation, many equivalents to the specific embodiments of the invention described herein. Therefore, the scope of the present invention is not intended to be limited to the above Description.

We claim:

1. A portable system for producing a formulation comprising lipid nanoparticle (LNP)-encapsulated RNA, the system comprising:
a first sub-system comprising multiple drug substance formulation modules, the first sub-system comprising:
a transcription module for forming an RNA solution via in vitro transcription; and
a second sub-system operatively downstream of the first sub-system comprising multiple drug product formation modules, the second sub-system comprising:
an LNP formulation module for producing a first RNA-LNP preparation from the RNA solution,
wherein each of the transcription module and the LNP formulation module is contained within a separate standard shipping container.

2. The system of claim 1, wherein each separate standard shipping container comprises a width of about 8 ft (2.43 m), a height of about 8.5 ft (2.59 m), and a length from about 20 ft (6.06 m) to about 40 ft (12.12 m).

3. The system of claim 1, wherein the LNP formulation module comprises at least one impingement jet mixing unit.

4. The system of claim 1, wherein the second sub-system further comprises a purification module disposed operatively downstream of the LNP formulation module, the purification module comprising at least one tangential flow filtration (TFF) unit, wherein the purification module is disposed within a separate standard shipping container.

5. The system of claim 1, wherein each of the first sub-system and the second sub-system comprises a bioburden reduction module disposed within a separate standard shipping container, each bioburden reduction module comprising a filtration unit comprising at least one filter with a pore size from about 0.05 µm to about 0.35 µm.

6. A portable LNP formulation system for producing a first RNA-LNP preparation comprising:
an impingement jet mixing unit; and
a tangential flow filtration (TFF) unit coupled fluidly downstream of the impingement jet mixing unit, the TFF unit for performing at least one diafiltration step and at least one ultrafiltration step,
wherein the system is disposed (and/or transported to a production site) within a single standard shipping container.

7. The system of claim 6, further comprising a bioburden reduction unit coupled fluidly downstream of the TFF unit and contained within the standard shipping container.

8. The system of claim 6, further comprising:
a first fluid conduit for delivering an RNA solution to the impingement jet mixing unit, the first fluid conduit fluidly connecting the impingement jet mixing unit to an RNA solution source external to the system;
a second fluid conduit for delivering a lipid solution to the impingement jet mixing unit, the second fluid conduit fluidly connecting the impingement jet mixing unit to a lipid solution source external to the system; and
a third fluid conduit for delivering a first RNA-LNP preparation to the TFF unit.

9. A drug production system comprising:
a drug substance module for producing at least one drug substance;
a drug product module for producing a drug product comprising the at least one drug substance;
wherein each of the drug substance module and the drug product module are disposed entirely in one or more portable shipping containers.

10. The system of claim 9, wherein each of the drug substance module and the drug product module are disposed within at least three (3) portable shipping containers.

11. The system of claim 9, wherein the drug product comprises at least one lipid nanoparticle (LNP).

12. The system of claim 9, further comprising at least one fill and finish module for disposing the drug product into at least one container.

13. The system of claim 9, wherein a combined power requirement of the drug substance module and the drug product module is in a range from about 200 kW to about 400 kW, with an uninterrupted power requirement in a range from about 50 kW to about 100 kW.

14. The system of claim 9, wherein a combined footprint of the drug substance module and the drug product module encompasses an area of from about 500 square meters to about 1000 square meters.

15. The system of claim 9, wherein each of the drug substance module and the drug product module comprises at least one airlock through which at least one of materials and personnel must pass when entering an operations area within the respective drug substance module and drug product module.

16. The system of claim 9, further comprising a quality control module comprising at least one of a PCR lab, an RNA/DNA lab, an environmental monitoring console, an HPLC lab, a cell culture lab, a general procedure lab, freezer monitoring equipment, a bioburden lab, a quality control storage area, a washing area, an endotoxin lab, and a gowning area.

17. The system of claim 9, further comprising at least one DNA sequencer for sequencing at least one local strain of a disease.

18. The system of claim 9, further comprising at least one DNA synthesizer for creating at least one custom DNA molecule.

19. The system of claim 9, further comprising at least one computing system for performing at least one of the following tasks:
uploading sequence information describing at least one local strain of a disease to a public database,
downloading sequence information describing the at least one local strain of a disease from the public database,
downloading DNA synthesis data to be used for making a vaccine that targets the at least one local strain of a disease from the public database, and
computing, based on the sequence information describing at least one local strain of a disease, a target strain upon which DNA synthesis data is based.

20. A method of producing a vaccine to treat a local strain of a disease, the method comprising:
filtering genomic data for the disease by at least one location, thereby producing localized data;
determining a target strain from the localized data;
sending DNA synthesis instructions to a site within, or proximate to, the at least one location;
producing, within, or proximate to, the at least one location, the vaccine to treat the local strain based on the DNA synthesis instructions.

21. The method of claim 20, wherein the disease is SARS-CoV-2.

22. The method of claim 20, further comprising at least one of administering the vaccine and distributing the vaccine within, or proximate to, the at least one location.

23. The method of claim 20, further comprising accessing a publically available database that houses the genomic data to be filtered.

24. The method of claim 20, further comprising:
sequencing, within, or proximate to, the at least one location, a sample of the local strain of the disease, thereby producing local strain sequence data; and
uploading the local strain sequence data to a publically available database that houses the genomic data to be filtered.

25. The method of claim 20, further comprising filtering the genomic data based on at least one of a range of dates and a lookback period,
wherein the range of dates and/or lookback period corresponds to a timeframe during which a localized outbreak of the disease occurred.

26. The method of claim 20, further comprising assessing deviations between the localized data and a baseline variant of the disease.

27. The method of claim 26, further comprising comparing the deviations for one or more subsets within the localized data.

28. The method of claim 27, further comprising assessing a level of commonality of the deviations for the one or more subsets within the localized data.

29. The method of claim 26, wherein determining a target strain from the localized data comprises determining a target strain based at least partially on the deviations between the localized data and the baseline variant of the disease.
